(19) United States
(12) Patent Application Publication
(10) Pub. No.: US 2025/0228606 A1
(43) Pub. Date: Jul. 17, 2025

(54) OPERATION PART FOR SURGICAL INSTRUMENT AND ELECTROCAUTERY SURGICAL INSTRUMENT EQUIPPED WITH OPERATION PART

(71) Applicant: LIVSMED INC., Seongnam-si, (KR)

(72) Inventors: Jae Yeong LEE, Seongnam-si, (KR); Hee Jin KIM, Seoul (KR); Jwa Nyun KIM, Yongin-si, (KR); Chung Hyun LIM, Seoul (KR)

(21) Appl. No.: 19/016,159

(22) Filed: Jan. 10, 2025

(30) Foreign Application Priority Data
Jan. 11, 2024 (KR) 10-2024-0004933
May 2, 2024 (KR) 10-2024-0058687

(51) Int. Cl. A61B 18/14; A61B 17/00; A61B 18/00

(52) U.S. Cl. CPC A61B 18/1445; A61B 2017/0042; A61B 2018/00595; A61B 2018/00601; A61B 2018/0063

(57) ABSTRACT

An operation part of a surgical instrument equipped with an end tool includes a handle, and an actuation operation part disposed on one side of the handle and configured to control an actuation motion of the end tool, which the actuation operation part includes an actuation lever rotatable around an actuation rotation shaft, an actuation pulley having one or more wires connected thereto coupled to the actuation lever, and rotatable in response to a rotation of the actuation lever, and an elastic member elastically deformably disposed between the actuation lever and the actuation pulley, and configured to transmit at least a portion of a rotational force of the actuation lever to the actuation pulley.

### CROSS-REFERENCE TO RELATED APPLICATION

This application is based on and claims priority under 35 U.S.C. § 119 to Korean Patent Application Nos. 10-2024-0004933, filed on Jan. 11, 2024, and 10-2024-0058687, filed on May 2, 2024, in the Korean Intellectual Property Office, the disclosure of which is incorporated by reference herein in its entirety.

### BACKGROUND

### 1. Field

The present disclosure relates to an electrocautery surgical instrument, and more particularly, to an electrocautery surgical instrument with improved insulation performance, capable of being mounted on a robot arm or operated manually for use in laparoscopic surgery or various other surgeries.

### 2. Description of the Related Art

In many cases, surgical procedures require the cutting and joining of body tissues, including organs, muscle tissue, connective tissue, and blood vessels. Over the centuries, sharp blades and sutures have been used for cutting and joining. However, during surgical procedures, cutting body tissues, especially those that are highly vascularized, results in bleeding. Accordingly, surgeons have sought surgical instruments and methods that can slow down or reduce bleeding during surgical procedures.

Recently, it has become possible to use electrosurgical instruments that utilize electrical energy for performing specific types of surgical tasks. For example, electrosurgical instruments equipped with one or more electrodes configured to supply electrical energy have been developed for use with surgical tools such as graspers, scissors, forceps, blades, needles, and hooks. Electrical energy supplied through the electrodes can be used to coagulate, join, or cut the patient's body tissues. In particular, when electrical energy is used, it becomes possible to perform cutting and hemostasis simultaneously.

Electrosurgical instruments are typically divided into two types, monopolar and bipolar. In the monopolar electrosurgical instrument, electrical energy of a specific polarity is supplied to one or more electrodes of the instrument. In addition, electrical energy of the opposite polarity is electrically connected to a patient. In the bipolar electrosurgical instrument, one or more electrodes are electrically connected to a first polarity electrical energy source, while one or more other electrodes are electrically connected to a second polarity electrical energy source opposite to the first polarity electrical energy source.

The background art described above is technical information retained by the present inventors in order to derive the present disclosure or obtained by the present inventors in the process of deriving the present disclosure, and thus is not necessarily known art disclosed to the general public before the filing of the present disclosure.

### SUMMARY

The present disclosure relates to a surgical instrument, which may be mounted on a robot arm or operated manually for use in laparoscopic surgery or various other surgeries and may include an elastic member capable of controlling a rotation of an actuation lever and an actuation pulley.

In an embodiment, an operation part of the surgical instrument may be equipped with an end tool, and the operation part may include a handle, and an actuation operation part disposed on one side of the handle and configured to control an actuation motion of the end tool, wherein the actuation operation part may include an actuation lever rotatable around an actuation rotation shaft, an actuation pulley having one or more wires positioned thereon coupled to the actuation lever and rotatable in response to a rotation of the actuation lever, and an elastic member elastically deformably disposed between the actuation lever and the actuation pulley, and configured to transmit at least a portion of a rotational force of the actuation lever to the actuation pulley.

In another embodiment of the present disclosure, the elastic member may be supported at one end by a pressing protrusion of the actuation lever and at another end by a support protrusion of the actuation pulley, and disposed with a predetermined restoring force between the pressing protrusion and the support protrusion.

In the other embodiment of the present disclosure, the elastic member may be configured to rotate in correspondence with the actuation lever and transmit the rotational force to the actuation pulley.

In the other embodiment of the present disclosure, when the rotational force is greater than a threshold value, the elastic member may be configured to elastically compress by a portion of a rotational force applied by the pressing protrusion and transmit a remaining portion of the rotational force to the actuation pulley.

In the other embodiment of the present disclosure, the elastic member may be configured to adjust, based on the predetermined restoring force, a rotational force received from the pressing protrusion when the actuation lever rotates.

In the other embodiment of the present disclosure, the elastic member may elastically deform when the rotational force, which is applied by the pressing protrusion when the actuation lever rotates, is greater than the predetermined restoring force.

In the other embodiment of the present disclosure, the actuation lever may include a pressing protrusion protruding toward the actuation pulley, the actuation pulley may include a support protrusion protruding toward the actuation lever, and the elastic member may be disposed between the pressing protrusion and the support protrusion.

In the other embodiment of the present disclosure, the actuation lever may include a plurality of pressing protrusions, wherein the plurality of pressing protrusions may be disposed spaced apart at preset intervals.

In the other embodiment of the present disclosure, when the actuation lever rotates, the actuation pulley may be configured to receive at least a portion of the rotational force of the actuation lever from the elastic member and transmit the at least a portion of the rotational force to the one or more wires.

In the other embodiment of the present disclosure, the actuation pulley may be configured to rotate in correspondence with the actuation lever and transmit the rotational force to the one or more wires.

In the other embodiment of the present disclosure, when the rotational force of the actuation lever is greater than a threshold value, the actuation pulley may be configured to receive a portion of the rotational force and transmit the portion of the rotational force to the one or more wires.

The present disclosure also relates to an electrocautery surgical instrument which may include an end tool that is rotatable in at least one direction, an operation part including a handle and an actuation operation part disposed on one side of the handle and configured to control an actuation motion of the end tool, and a power transmission part including an actuation wire connecting the end tool to the actuation operation part, and configured to transmit power from the actuation operation part to the end tool, wherein the actuation operation part may include an actuation lever rotatable around an actuation rotation shaft, an actuation pulley having the actuation wire positioned thereon coupled to the actuation lever, and rotatable in response to a rotation of the actuation lever, and an elastic member elastically deformably disposed between the actuation lever and the actuation pulley, and configured to transmit at least a portion of a rotational force of the actuation lever to the actuation pulley.

In the other embodiment of the present disclosure, the elastic member may be supported at one end by a pressing protrusion of the actuation lever and at another end by a support protrusion of the actuation pulley, and disposed with a predetermined restoring force between the pressing protrusion and the support protrusion.

In the other embodiment of the present disclosure, the elastic member may be configured to rotate in correspondence with the actuation lever and transmit the rotational force to the actuation pulley.

In the other embodiment of the present disclosure, when the rotational force is greater than a threshold value, the elastic member may be configured to elastically compress by a portion of a rotational force applied by the pressing protrusion and transmit a remaining portion of the rotational force to the actuation pulley.

In the other embodiment of the present disclosure, the elastic member may be configured to adjust, based on the predetermined restoring force, a rotational force received from the pressing protrusion when the actuation lever rotates.

In the other embodiment of the present disclosure, the elastic member may be configured to elastically deform when the rotational force, which is applied by the pressing protrusion when the actuation lever rotates, is greater than the predetermined restoring force.

In the other embodiment of the present disclosure, the actuation lever may include a pressing protrusion protruding toward the actuation pulley, the actuation pulley may include a support protrusion protruding toward the actuation lever, and the elastic member may be disposed between the pressing protrusion and the support protrusion.

In the other embodiment of the present disclosure, the actuation lever may include a plurality of pressing protrusions, wherein the plurality of pressing protrusions may be disposed spaced apart at preset intervals.

In the other embodiment of the present disclosure, when the actuation lever rotates, the actuation pulley may receive at least a portion of the rotational force of the actuation lever from the elastic member and transmit the at least a portion of the rotational force to the actuation wire.

In an embodiment of the present disclosure, the actuation pulley may rotate in correspondence with the actuation lever and transmits the rotational force to the actuation wire.

In an embodiment of the present disclosure, the actuation pulley may receive a portion of the rotational force and transmits the portion of the rotational force to the actuation wire, when the rotational force of the actuation lever is greater than a threshold value.

Other aspects, features, and advantages beyond those described above will become apparent from the following drawings, claims, and detailed description of the disclosed embodiments.

### BRIEF DESCRIPTION OF THE DRAWINGS

The aforementioned and other aspects, features, and advantages of certain embodiments of the present disclosure will be more apparent from the following description taken in conjunction with the accompanying drawings, in which:

FIG. 1 is a perspective view illustrating an electrocautery surgical instrument according to an embodiment of the present disclosure.

FIG. 2 is a perspective view illustrating the electrocautery surgical instrument of FIG. 1;

FIGS. 3 and 4 are perspective views illustrating an operation part of the electrocautery surgical instrument of FIG. 1;

FIG. 5 is a view schematically illustrating only a configuration of pulleys and wires constituting joints of the electrocautery surgical instrument of FIG. 1;

FIG. 6 is a perspective view illustrating a yaw motion of the electrocautery surgical instrument of FIG. 2;

FIGS. 7 and 8 are diagrams illustrating a first jaw and a second jaw, respectively, of the configuration of pulleys and wires associated with an actuation motion and the yaw motion of the electrocautery surgical instrument illustrated in FIG. 1;

FIG. 9 is a perspective view illustrating a pitch motion of the electrocautery surgical instrument of FIG. 1;

FIGS. 10 and 11 are diagrams illustrating the first jaw and the second jaw, respectively, of the configuration of pulleys and wires associated with the pitch motion of the electrocautery surgical instrument illustrated in FIG. 1;

FIGS. 12 and 13 are perspective views illustrating an operation of an actuation lever of the electrocautery surgical instrument illustrated in FIG. 1;

FIGS. 14A to 15B are views illustrating the movement of wires during the operation of the actuation lever of the electrocautery surgical instrument illustrated in FIG. 1;

FIGS. 16 and 17 are exploded perspective views of the actuation lever and an actuation pulley of the electrocautery surgical instrument of FIG. 1; and

FIGS. 18 to 20 are views illustrating an operation process of the actuation lever of the electrocautery surgical instrument of FIG. 1.

### DETAILED DESCRIPTION OF DISCLOSED EMBODIMENTS

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will be clarified by the following description along with the accompanying drawings. However, the embodiments are not limited to the embodiments disclosed below, but may be implemented in various forms.

In describing the present disclosure, a detailed description of known related arts will be omitted when it is determined that the essence of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and do not preclude the possibility of adding one or more other features or components.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude other members from being interposed between the two members.

Sizes of components in the drawings may be exaggerated or reduced for convenience of description. For example, since the size and thickness of each component shown in the drawings are arbitrarily presented for convenience of description, the following embodiments are not necessarily limited thereto.

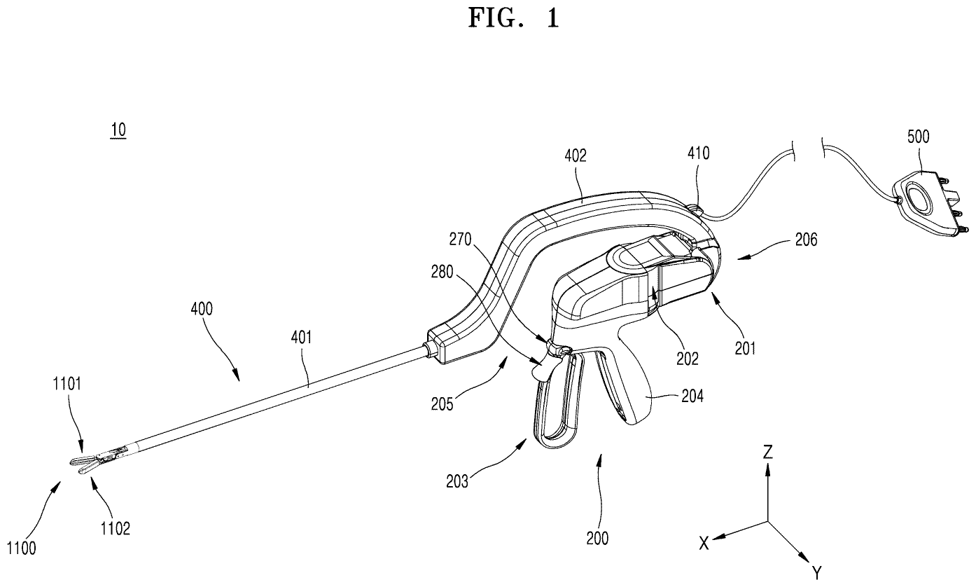

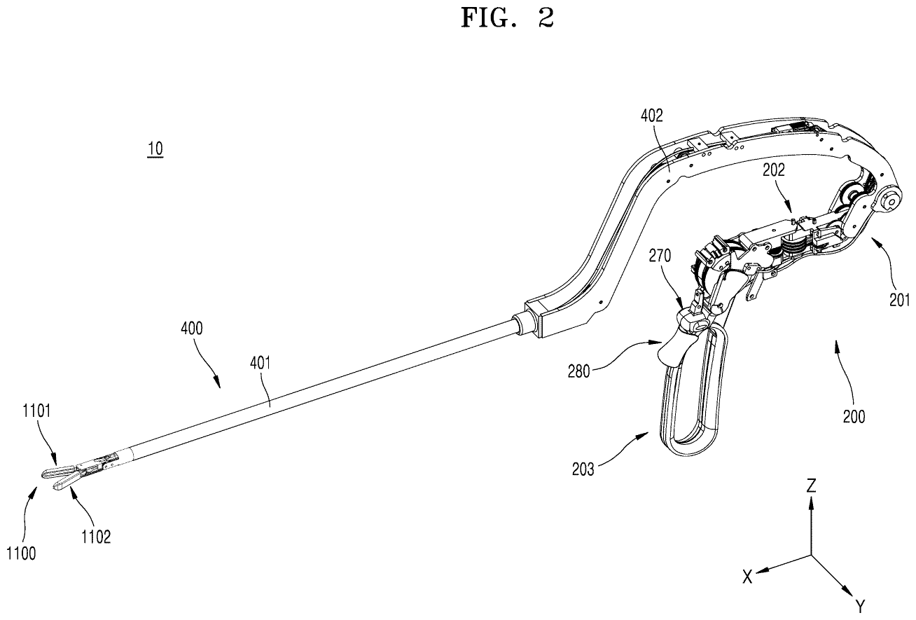

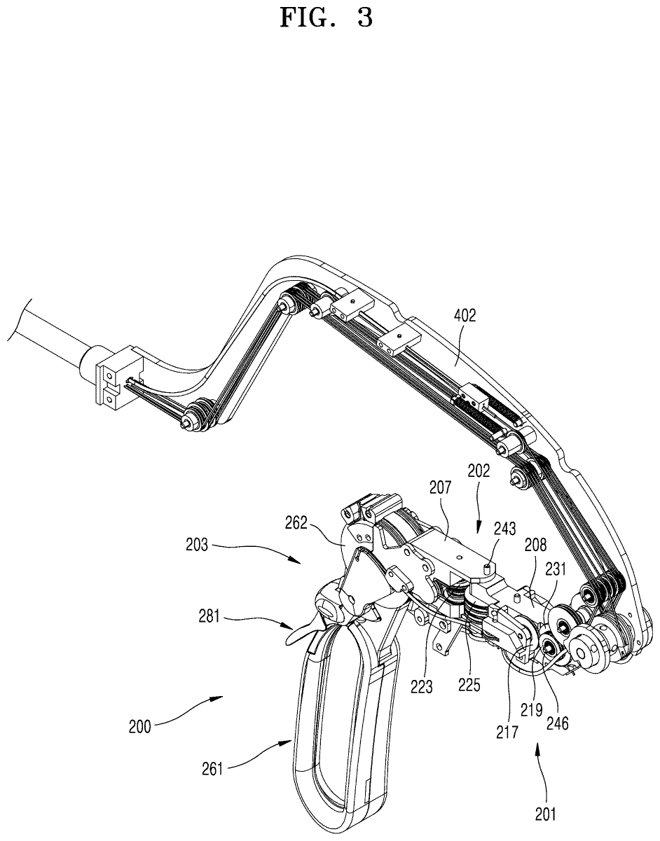

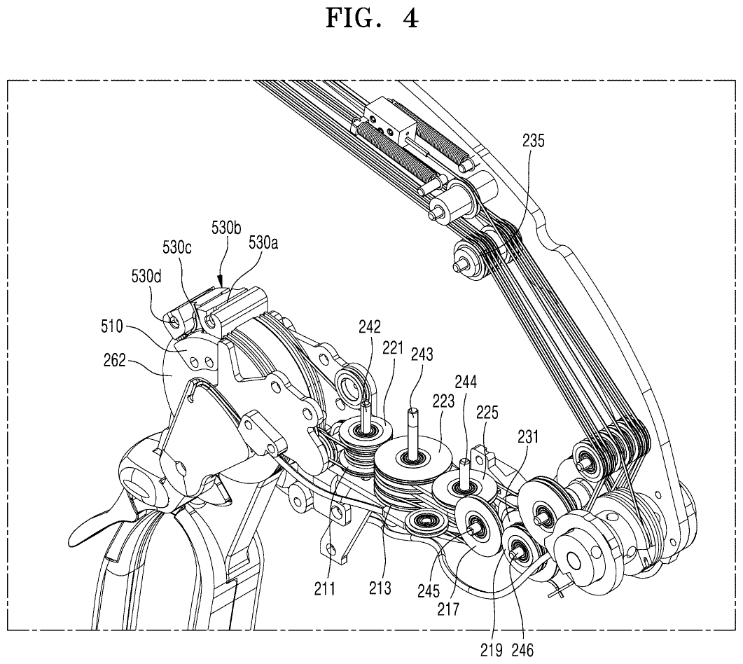

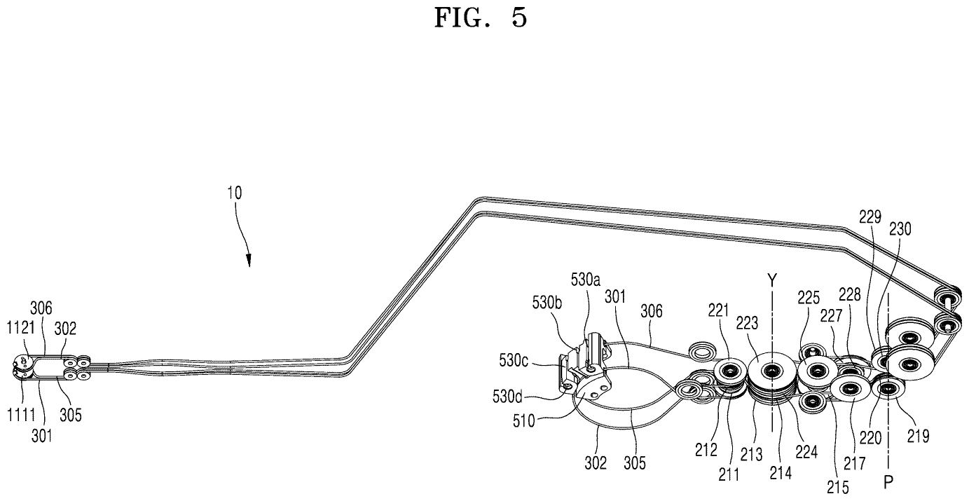

FIG. 1 is a perspective view illustrating an electrocautery surgical instrument according to an embodiment of the present disclosure. FIG. 2 is a perspective view illustrating the electrocautery surgical instrument of FIG. 1, and FIGS. 3 and 4 are perspective views illustrating an operation part of the electrocautery surgical instrument of FIG. 1. In addition, FIG. 5 is a view schematically illustrating only a configuration of pulleys and wires constituting joints of the electrocautery surgical instrument of FIG. 1.

Referring to FIGS. 1 to 5, an electrocautery surgical instrument 10 according to an embodiment of the present disclosure includes an end tool 1100, an operation part 200, a power transmission part 300, and a connection part 400.

The connection part 400 may be formed in a shape of a hollow shaft, and at least one or more wires and electric wires may be accommodated therein. The operation part 200 may be coupled to one end portion of the connection part 400, the end tool 1100 may be coupled to another end portion of the connection part 400, and the connection part 400 may connect the operation part 200 to the end tool 1100. Here, the connection part 400 of the electrocautery surgical instrument 10 according to an embodiment of the present disclosure may include a straight part 401 and a curved part 402, in which the straight part 401 is disposed at a side of the connection part 400 coupled to the end tool 1100, and the curved part 402 is formed at a side of the connection part 400 to which the operation part 200 is coupled. As such, since the end portion of the connection part 400 at the side of the operation part 200 may be formed to be bent, a pitch operation part 201, a yaw operation part 202, and an actuation operation part 203 may be formed along or adjacent to an extension line of the end tool 1100. In other words, the pitch operation part 201 and the yaw operation part 202 may at least partially be accommodated in a concave portion formed by the curved part 402. Due to such shape of the curved part 402, the shapes and motions of the operation part 200 and the end tool 1100 may be more intuitively consistent.

In another embodiment, a plane on which the curved part 402 is formed may be substantially the same as a pitch plane, that is, an XZ plane of FIG. 2. As such, as the curved part 402 is formed on the plane substantially the same as the XZ plane, the interference with the operation part 200 may be reduced. For intuitive motions of the end tool and the operation part, configurations other than the XZ plane are also possible.

In the other embodiment, a connector 410 may be formed on the curved part 402. The connector 410 may be connected to an external power supply 500, and the connector 410 may be connected to jaws 1103 through electric wires 411 and 412 to transfer electrical energy supplied from the external power supply 500 to the jaws 1103. Here, the connector 410 may be of a bipolar-type with two electrodes or a monopolar type with one electrode.

The operation part 200 may be disposed at the one end portion of the connection part 400 and provided as an interface to be directly controlled by a medical doctor, for example, a tongs shape, a stick shape, a lever shape, or the like, and when the medical doctor controls the operation part 200, the end tool 1100, which is connected to the interface and inserted into the body of a surgical patient, performs a certain motion, thereby performing surgery. Here, the operation part 200 is illustrated in FIG. 2 as being formed in a handle shape that is rotatable while the finger is inserted therein, but the principle of the present disclosure is not limited thereto, and various forms of operation part 200 may be possible for it to connect to the end tool 1100 and operate the end tool 1100.

The end tool 1100 is formed on the other end portion of the connection part 400, and performs necessary motions for surgery by being inserted into a surgical site. In an example of the end tool 1100 described above, as shown in FIG. 2, the end tool 1100 may include a pair of jaws for performing a grip motion, that is, a first jaw 1101 and a second jaw 1102. Here, each of the first jaw 1101 and the second jaw 1102, or a component encompassing the first jaw 1101 and the second jaw 1102 may be referred to as the jaws 1103.

However, the principle of the present disclosure is not limited thereto, and various devices for performing surgery may be used as the end tool 1100. For example, a configuration such as a cantilever cautery may also be used as the end tool 1100. The end tool 1100 is connected to the operation part 200 by the power transmission part 300, and receives a driving force of the operation part 200 through the power transmission part 300 to perform a motion necessary for surgery, such as gripping, cutting, suturing, or the like.

Here, the end tool 1100 of the electrocautery surgical instrument 10 according to an embodiment of the present disclosure may be formed to be rotatable in at least one direction, for example, the end tool 1100 may perform a pitch motion around a Y-axis of FIG. 2 and simultaneously perform a yaw motion and an actuation motion around a Z-axis of FIG. 2. Here, the actuation motion refers to a motion in which the first jaw 1101 and the second jaw 1102 of the end tool 1100 rotate in opposite directions. That is, the end tool 1100 may perform a grip motion or release the grip motion through the actuation motion.

In addition, the end tool 1100 may include a first jaw pulley 1111 or the like associated with a rotational motion of the first jaw 1101. In addition, the end tool 1100 may include a second jaw pulley 1121 or the like associated with a rotational motion of the second jaw 1102.

The power transmission part 300 may connect the operation part 200 to the end tool 1100, transmit the driving force of the operation part 200 to the end tool 1100, and include a plurality of wires, pulleys, links, sections, gears, and the like.

Referring to FIG. 5, the power transmission part 300 of the electrocautery surgical instrument 10 according to an embodiment of the present disclosure may include a first wire 301, a second wire 302, a third wire 303, a fourth wire 304, a fifth wire 305, and a sixth wire 306.

Here, the first wire 301 and the fifth wire 305 may be paired to serve as first jaw wires. The second wire 302 and the sixth wire 306 may be paired to serve as second jaw wires. Here, the components encompassing the first wire 301 and the fifth wire 305, which are the first jaw wires, and the second wire 302 and the sixth wire 306, which are the second jaw wires, may be referred to as jaw wires. In addition, the third wire 303 and the fourth wire 304 may be paired to serve as pitch wires.

In addition, the power transmission part 300 of the electrocautery surgical instrument 10 according to an embodiment of the present disclosure may include a fastening member (not shown) coupled to an end portion of each of the wires in order to couple the wires to the pulleys. Here, each of the fastening members may have various shapes as necessary, such as a ball shape, a tube shape, and the like.

The coupling relationship between the wires, the fastening members, and the respective pulleys will be described in detail as follows.

First, the first wire 301 and the fifth wire 305, which are first jaw wires, may be a single wire. The fastening member (not shown), which is a first jaw wire-end tool fastening member, is inserted at an intermediate point of the first jaw wire, which is a single wire, and the fastening member (not shown) is crimped and fixed, and then, both strands of the first jaw wire centered on the fastening member (not shown) may be referred to as the first wire 301 and the fifth wire 305, respectively.

Alternatively, the first wire 301 and the fifth wire 305, which are first jaw wires, may also be formed as separate wires and connected by the fastening member (not shown).

In addition, by coupling the fastening member (not shown) to the first jaw pulley 1111, the first wire 301 and the fifth wire 305 may be fixedly coupled to the first jaw pulley 1111. This allows the first jaw pulley 1111 to rotate as the first wire 301 and the fifth wire 305 are pulled and released.

In the other embodiment, a first jaw wire-operation part fastening member may be coupled to another end portions of the first wire 301 and the fifth wire 305, which are opposite to one end portions to which the first jaw wire-end tool fastening member is fastened.

In addition, by coupling the first jaw wire-operation part fastening member to a first pulley 211, the first wire 301 and the fifth wire 305 may be fixedly coupled to the pulley 211. As a result, when the first pulley 211 may be rotated by a motor or human power, the first wire 301 and the fifth wire 305 are pulled and released, allowing the first jaw pulley 1111 of the end tool 1100 to rotate.

In the same manner, the second wire 302 and the sixth wire 306, which are second jaw wires, may respectively be coupled to a second jaw wire-end tool fastening member and a second jaw wire-operation part fastening member. In addition, the second jaw wire-end tool fastening member may be coupled to the second jaw pulley 1121, and the second jaw wire-operation part fastening member is coupled to a tenth pulley 220. As a result, when the tenth pulley 220 is rotated by a motor or a human force, the second jaw pulley 1121 of the end tool 1100 may be rotated as the second wire 302 and the sixth wire 306 are pulled and released.

In the same manner, the third wire 303 and the fourth 304, which are pitch wires, may respectively be coupled to a pitch wire-end tool fastening member and a pitch wire-operation part fastening member.

### Operation Part

Hereinafter, the operation part 200 of the electrocautery surgical instrument 10 of FIG. 1 will be described in more detail.

Referring to FIGS. 1 to 5, the operation part 200 of the electrocautery surgical instrument 10 according to an embodiment of the present disclosure may include a handle 204 that a user can grip, the actuation operation part 203 configured to control an actuation motion of the end tool 1100, the yaw operation part 202 configured to control a yaw motion of the end tool 1100, and the pitch operation part 201 configured to control a pitch motion of the end tool 1100. Here, it is understood that only the components related to the pitch/yaw/actuation motions of the electrocautery surgical instrument 10 are illustrated in FIGS. 3 and 4.

In addition, the operation part 200 of the electrocautery surgical instrument 10 further includes a blade operation part 260 that controls the movement of a blade of the end tool 1100 to perform cutting, and a sealing operation part 270 that controls the supply of electric energy to electrodes (not shown) of the end tool 1100 to perform cauterization.

The operation part 200 may include the first pulley 211, a second pulley 212, a third pulley 213, a fourth pulley 214, a fifth pulley 215, a seventh pulley 217, an eighth pulley 218, a ninth pulley 219, and the tenth pulley 220 that are related to the rotational motion of the first jaw 1101. In addition, the operation part 200 may include an eleventh pulley 221, a twelfth pulley 222, a thirteenth pulley 223, a fourteenth pulley 224, a fifteenth pulley 225, a seventeenth pulley 227, an eighteenth pulley 228, a nineteenth pulley 229, and a twentieth pulley 230, which are related to the rotational motion of the second jaw 1102. In addition, the operation part 200 may include the actuation pulley 262 associated with the rotational motion of the first jaw 1101 and the second jaw 1102. In addition, the operation part 200 may include a twenty-first pulley 231 associated with a pitch motion. In addition, the operation part 200 may include at least one relay pulley 235 disposed at some places along the curved part 402 of the connection part 400.

Here, the pulleys facing each other are illustrated in the drawings as being formed parallel to each other, but the principle of the present disclosure is not limited thereto, and each of the pulleys may be formed in various positions and sizes suitable for the configuration of the operation part.

Further, the operation part 200 according to an embodiment of the present disclosure may include a first rotation shaft 241, a second rotation shaft 242, a third rotation shaft 243, a fourth rotation shaft 244, a fifth rotation shaft 245, and a sixth rotation shaft 246. Here, the first rotation shaft 241 may function as an operation part actuation rotation shaft, and the second rotation shaft 242 may function as an operation part first yaw sub-rotation shaft. In addition, the third rotation shaft 243 may function as an operation part yaw main rotation shaft, and the fourth rotation shaft 244 may function as an operation part second yaw sub-rotation shaft. In addition, the fifth rotation shaft 245 may function as an operation part pitch sub-rotation shaft, and the sixth rotation shaft 246 may function as an operation part pitch main rotation shaft.

The first rotation shaft 241, the second rotation shaft 242, the third rotation shaft 243, the fourth rotation shaft 244, the fifth rotation shaft 245, and the sixth rotation shaft 246 may be arranged sequentially from a distal end 205 toward a proximal end 206 of the operation part 200.

The actuation pulley 262 functions as an actuation pulley of the first jaw 1101 and the second jaw 1102, and may be referred to as an operation part actuation pulley.

The first pulley 211 and the second pulley 212 function as operation part first-jaw first-yaw-sub-pulleys, the eleventh pulley 221 and the twelfth pulley 222 function as operation part second-jaw first-yaw-sub-pulleys, and these components may also be collectively referred to as an operation part first yaw sub-pulley.

The third pulley 213 and the fourth pulley 214 function as operation part first-jaw yaw main pulleys, the thirteenth pulley 223 and the fourteenth pulley 224 function as operation part second-jaw yaw main pulleys, and these components may also be collectively referred to as an operation part yaw main pulley.

The fifth pulley 215 functions as an operation part first-jaw second-yaw-sub-pulley, the fifteenth pulley 225 functions as an operation part second-jaw second-yaw-sub-pulley, and these components may also be collectively referred to as an operation part second yaw sub-pulley.

The seventh pulley 217 and the eighth pulley 218 function as operation part first-jaw pitch sub-pulleys, the seventeenth pulley 227 and the eighteenth pulley 228 function as operation part second-jaw pitch sub-pulleys, and these components may also be collectively referred to as an operation part pitch sub-pulley.

The ninth pulley 219 and the tenth pulley 220 function as operation part first-jaw pitch main pulleys, and the nineteenth pulley 229 and the twentieth pulley 230 function as operation part second-jaw pitch main pulleys, and these components may also be collectively referred to as an operation part pitch main pulley.

The twenty-first pulley 231 functions as an operation part pitch wire main pulley, and may include a pulley (not shown) that functions as an operation part pitch wire sub-pulley.

The above components are categorized from the perspective of the operation part 200 for each motion (pitch/yaw/actuation) as follows.

The pitch operation part 201 configured to control a pitch motion of the end tool 1100 may include the seventh pulley 217, the eighth pulley 218, the ninth pulley 219, the tenth pulley 220, the seventeenth pulley 227, the eighteenth pulley 228, the nineteenth pulley 229, the twentieth pulley 230, and the twenty-first pulley 231. In addition, the pitch operation part 201 may include the fifth rotation shaft 245 and the sixth rotation shaft 246. In addition, the pitch operation part 201 may further include a pitch frame 208.

The yaw operation part 202 configured to control a yaw motion of the end tool 1100 may include the first pulley 211, the second pulley 212, the third pulley 213, the fourth pulley 214, the fifth pulley 215, the eleventh pulley 221, the twelfth pulley 222, the thirteenth pulley 223, the fourteenth pulley 224, and the fifteenth pulley 225. In addition, the yaw operation part 202 may include the second rotation shaft 242, the third rotation shaft 243, and the fourth rotation shaft 244. In addition, the yaw operation part 202 may further include a yaw frame 207.

The actuation operation part 203 configured to control an actuation motion of the end tool 1100 may include the actuation pulley 262 and the first rotation shaft 241.

Hereinafter, each component of the operation part 200 will be described in more detail.

The handle 204 may be formed to be gripped by a user with the hand, and more specifically, may be formed to be grasped by the user by wrapping the handle 204 with his/her palm. In addition, the actuation operation part 203 and the yaw operation part 202 are formed on the handle 204, and the pitch operation part 201 is formed on one side of the yaw operation part 202. In addition, one end portion of the pitch operation part 201 is connected to the curved part 402 of the connection part 400.

The actuation operation part 203 includes a blade operation part 260. The blade operation part 260 may include an actuation lever 261 and the actuation pulley 262.

Here, the actuation lever 261 is formed in the shape of a hand ring, which the user may operate by inserting his or her finger therein.

Here, the first rotation shaft 241, which is an actuation rotation shaft, may be formed to form a certain angle with the XZ plane on which the connection part 400 is formed.

For example, the first rotation shaft 241 may be formed in a direction parallel to the Y-axis, and in this state, when the pitch operation part 201 or the yaw operation part 202 is rotated, a coordinate system of the actuation operation part 203 may change relatively. However, the principle of the present disclosure is not limited thereto, and the rotation shaft 241 may be formed in various directions according to ergonomic design principles depending on the hand structure of the user gripping the actuation operation part 203.

In the other embodiment, the actuation pulley 262 and the actuation lever 261 may be coupled to each other or may be formed as a single member. Accordingly, the actuation pulley 262 may rotate along with the rotation of the actuation lever 261.

Here, the actuation pulley 262 may be configured as a single pulley or two pulleys fixedly coupled to each other.

The specific shape and arrangement of the actuation lever 261 and the actuation pulley 262 will be described in detail below.

The yaw operation part 202 may include the second rotation shaft 242, the third rotation shaft 243, the third pulley 213 and the fourth pulley 214, which are operation part first-jaw yaw main pulleys, the thirteenth pulley 223 and the fourteenth pulley 224, which are operation part second-jaw yaw main pulleys, and the yaw frame 207. In addition, the yaw operation part 202 may further include the first pulley 211 and the second pulley 212, which are operation part first-jaw first-yaw-sub-pulleys formed on one side of the third pulley 213 and one side of the fourth pulley 214, respectively, and the eleventh pulley 221 and the twelfth 222, which are operation part second-jaw first-yaw-sub-pulleys formed on one side of the eleventh pulley 221 and one side of the twelfth pulley 222, respectively. In addition, the yaw operation part 202 may further include the fifth pulley 215, which is an operation part first-jaw second-yaw-sub-pulley formed on another side of the third pulley 213 and the fourth pulley 214, and the fifth pulley 225 which is an operation part second-jaw second-yaw-sub-pulley formed on another side of the thirteenth pulley 223 and the fourteenth pulley 224. Here, the fifth pulley 215 and the fifteenth pulley 225 may be coupled to the pitch frame 208 which is described below.

Here, it is illustrated in the drawings that the yaw operation part 202 includes the third pulley 213 and the fourth pulley 214 and the thirteenth pulley 223 and the fourteenth pulley 224, in which the third pulley 213 and the fourth pulley 214 and the thirteenth pulley 223 and the fourteenth pulley 224 each may include two pulleys that are facing each other and independently rotatable, but the principle of the present disclosure is not limited thereto. That is, one or more pulleys having the same diameter or different diameters may be provided according to the configuration of the yaw operation part 202.

In detail, the second rotation shaft 242, which is an operation part first yaw sub-rotation shaft, is formed on one side of the actuation operation part 203 on the handle 204, and the third rotation shaft 243, which is an operation part yaw main rotation shaft, is formed on one side of the second rotation shaft 242. At this time, the handle 204 is formed to be rotatable around the third rotation shaft 243.

Here, the third rotation shaft 243 may be formed to form a certain angle with an XY plane on which the connection part 400 is formed. For example, the third rotation shaft 243 may be formed in a direction parallel to the Z-axis, and in this state, when the pitch operation part 201 is rotated, a coordinate system of the third rotation shaft 243 may change accordingly. Of course, the principle of the present disclosure is not limited thereto, and the third rotation shaft 243 may be formed in various directions according to the ergonomic design principles depending on the hand structure of the user gripping the operation part 200.

In the other embodiments, the third pulley 213 and the fourth pulley 214 and the thirteenth pulley 223 and the fourteenth pulley 224 may be coupled to the third rotation shaft 243 configured to be rotatable around the third rotation shaft 243. In addition, the first wire 301 or the fifth wire 305, which are the first jaw wires, is wound around the third pulley 213 and the fourth 214, and the second wire 302 or the sixth wire 306, which are the second jaw wires, may be wound around the thirteenth pulley 223 and the fourteenth pulley 224. In this case, the third pulley 213 and the fourth pulley 214 and the thirteenth pulley 223 and the fourteenth pulley 224 may each include two pulleys that are facing each other and independently rotatable. Accordingly, a wire being wound and a wire being unwound may each be wound around separate pulleys so that the wires may perform motions without interfering with each other.

The yaw frame 207 rigidly connects the handle 204, the second rotation shaft 242, and the third rotation shaft 243 to each other, and the actuation operation part 203, to which the first rotation shaft 241 and the actuation pulley 262 are coupled, is rigidly connected to the yaw frame 207 either directly or through a relay member, thereby allowing the handle 204, the yaw operation part 202, and the actuation operation part 203 to integrally yaw-rotate around the third rotation shaft 243.

The pitch operation part 201 may include the sixth rotation shaft 246, the ninth pulley 219 and the tenth pulley 220, which are operation part first-jaw pitch main pulleys, the nineteenth pulley 229 and the twentieth pulley 230, which are operation part second-jaw pitch main pulleys, and the pitch frame 208. In addition, the pitch operation part 201 may further include the fifth rotation shaft 245, the seventh pulley 217 and the eighth pulley 218, which are operation part first-jaw pitch sub-pulleys formed on one side of the ninth pulley 219 and one side of the tenth pulley 220, respectively, and the seventeenth pulley 227 and the eighteenth pulley 228, which are operation part second-jaw pitch sub-pulleys formed on one side of the nineteenth pulley 229 and one side of the twentieth pulley 230, respectively. The pitch operation part 201 may be connected to the curved part 402 of the connection part 400 through the sixth rotation shaft 246.

More specifically, the pitch frame 208 is a base frame of the pitch operation part 201, and the third rotation shaft 243 is rotatably coupled to one end portion of the pitch frame 208. That is, the yaw frame 207 is formed to be rotatable around the third rotation shaft 243 with respect to the pitch frame 208.

As described above, since the yaw frame 207 connects the handle 204, the third rotation shaft 243, the first rotation shaft 241, and the second rotation shaft 242 to each other, and the yaw frame 207 is also axially coupled to the pitch frame 208, when the pitch frame 208 is pitch-rotated around the sixth rotation shaft 246, the yaw frame 207 connected to the pitch frame 208, the handle 204, the first rotation shaft 241, the second rotation shaft 242, and the third rotation shaft 243 are pitch-rotated together with the pitch frame 208. That is, when the pitch operation part 201 is rotated around the sixth rotation shaft 246, the actuation operation part 203 and the yaw operation part 202 are rotated together with the pitch operation part 201. In other words, when a user pitch-rotates the handle 204 around the sixth rotation shaft 246, the actuation operation part 203, the yaw operation part 202, and the pitch operation part 201 are moved together with the handle 204.

The ninth pulley 219 and the tenth pulley 220 and the nineteenth pulley 229 and the twentieth pulley 230 may be coupled to the sixth rotation shaft 246 so as to be rotatable around the sixth rotation shaft 246 of the pitch frame 208.

Here, the ninth pulley 219 and the tenth pulley 220 may be formed to face each other so as to be independently rotatable. Accordingly, a wire being wound and a wire being unwound may each be wound around separate pulleys so that the wires may perform motions without interfering with each other. Similarly, the nineteenth pulley 229 and the twentieth pulley 230 may also be formed to face each other so as to be independently rotatable. Accordingly, a wire being wound and a wire being unwound may each be wound around separate pulleys so that the wires may perform motions without interfering with each other.

A structural connection between each of the handle 204, the pitch operation part 201, the yaw operation part 202, and the actuation operation part 203 is summarized as follows. The first rotation shaft 241, the second rotation shaft 242, the third rotation shaft 243, the fourth rotation shaft 244, the fifth rotation shaft 245, and the sixth rotation shaft 246 may be formed on the handle 204. In addition, since the second rotation shaft 242 and the third rotation shaft 243 are directly formed on the handle 204, the handle 204 and the yaw operation part 202 may be directly connected to each other. On the other hand, since the pitch operation part 201 is formed on the one side of the yaw operation part 202 so as to be connected to the yaw operation part 202, the pitch operation part 201 may be configured to be indirectly connected to the handle 204 through the yaw operation part 202, rather than being directly connected to the handle 204. In addition, since the actuation operation part 203 is formed on another side of the yaw operation part 202 so as to be connected to the yaw operation part 202, the actuation operation part 203 may be configured to be indirectly connected to the handle 204 through the yaw operation part 202, rather than being directly connected to the handle 204.

In the electrocautery surgical instrument 10 according to an embodiment of the present disclosure, the pitch operation part 201 and the end tool 1100 may be formed on the same axis (X-axis) or on parallel axes. That is, the sixth rotation shaft 246 of the pitch operation part 201 is formed at one end portion of the curved part 402 of the connection part 400, and the end tool 1100 is formed at the other end portion of the connection part 400.

In addition, the at least one relay pulley 235 configured to change or guide paths of the wires may be positioned at some places along the connection part 400, particularly in the curved part 402. As at least some of the wires are wound around the at least one relay pulley 235 configured to guide the paths of the wires, these wires may be positioned along a bent shape of the curved part 402.

It is illustrated that the connection part 400 may be formed in a curved shape with a certain curvature by having the curved part 402, but the principle of the present disclosure is not limited thereto, and the connection part 400 may be formed linearly or shaped to be bent one or more times as necessary, and even in this case, the pitch operation part 201 and the end tool 1100 may be formed on substantially the same axis or parallel axes. In addition, although FIG. 2 illustrates that each of the pitch operation part 201 and the end tool 1100 is formed on an axis parallel to the X-axis, the principle of the present disclosure is not limited thereto, and the pitch operation part 201 and the end tool 1100 may be formed on different axes.

### Actuation, Yaw, and Pitch Motions

The actuation motion, the yaw motion, and the pitch motion of the present embodiment will be described in detail below.

First, the actuation motion will be described below.

When a user inserts his or her finger into a finger ring formed on the actuation lever 261 and rotates the actuation lever 261 using his or her finger, the actuation pulley 262 coupled to the actuation lever 261 may rotate around the first rotation shaft 241.

In this case, the first wire 301 and the fifth wire 305, each having one end portion fixedly coupled to and wound around the actuation pulley 262, and the second wire 302 and the sixth wire 306, each having one end portion fixedly coupled to and wound around the same actuation pulley 262, move as the actuation pulley 262 rotates. Here, although the first wire 301, the second wire 302, the fifth wire 305, and the sixth wire 306 are all coupled to the same actuation pulley 262, the movement of each wire may vary depending on the direction in which each wire is wound around the actuation pulley 262 as the pulley rotates. This mechanism will be described in detail below.

In addition, a rotating force is transmitted to the end tool 1100 through the power transmission part 300, and the jaws 1103 of the end tool 1100 may perform the actuation motion.

Here, as described above, the actuation motion refers to a motion in which the first jaw 1101 and the second jaw 1102 are splayed or closed while being rotated in opposite directions. That is, when the actuation lever 261 of the actuation operation part 203 is rotated in a direction close to the handle 204, the first jaw 1101 is rotated in a counterclockwise direction, and the second jaw 1102 is rotated in a clockwise direction, thereby closing the end tool 1100. On the contrary, when the actuation lever 261 of the actuation operation part 203 is rotated in a direction away from the handle 204, the first jaw 1101 is rotated in the clockwise direction, and the second jaw 1102 is rotated in the clockwise direction, thereby opening the end tool 1100.

Next, the yaw motion will be described below.

When a user rotates the handle 204 around the third rotation shaft 243 while holding the handle 204, the actuation operation part 203 and the yaw operation part 202 are yaw-rotated around the third rotation shaft 243. That is, when the actuation pulley 262, to which the first wire 301 and fifth wire 305 are fixedly coupled, is rotated around the third rotation shaft 243, the first wire 301 and the fifth wire 305 wound around the third pulley 213 and the fourth pulley 214 are moved. Similarly, since the second wire 302 and the sixth wire 306 are also fixedly coupled to the actuation pulley 262, when the actuation pulley 262 rotates around the third rotation shaft 243, the second wire 302 and the sixth wire 306 wound around the thirteenth pulley 223 and the fourteenth pulley 224 may be moved. In this case, the first wire 301 and the fifth wire 305 connected to the first jaw 1101 and the second wire 302 and the sixth wire 306 connected to the second jaw 1102 are wound around the third pulley 213 and the fourth pulley 214 and the pulley thirteenth pulley 223 and the fourteenth pulley 224, so that the first jaw 1101 and the second jaw 1102 are rotated in the same direction during the yaw rotation. In addition, as a rotating force is transmitted to the end tool 1100 through the power transmission part 300, the yaw motion in which the jaws 1103 of the end tool 1100 are rotated in the same direction is performed.

In this case, since the yaw frame 207 connects the handle 204, the first rotation shaft 241, the second rotation shaft 242, and the third rotation shaft 243 to each other, the handle 204, the yaw operation part 202, and the actuation operation part 203 are rotated together around the third rotation shaft 243.

Next, the pitch motion will be described below.

When a user rotates the handle 204 around the sixth rotation shaft 246 while holding the handle 204, the actuation operation part 203, the yaw operation part 202, and the pitch operation part 201 are pitch-rotated around the rotation shaft 246. That is, when the actuation pulley 262, to which the first wire 301 and the fifth wire 305 are fixedly coupled, is rotated around the sixth rotation shaft 246, the first wire 301 and the fifth wire 305 wound around the ninth pulley 219 and the tenth pulley 220 are moved. Similarly, when the actuation pulley 262, to which the second wire 302 and the sixth wire 306 are fixedly coupled, is rotated around the sixth rotation shaft 246, the second wire 302 and the sixth wire 306 wound around the nineteenth pulley 229 and the twentieth pulley 230 are moved. In this case, as described with reference to FIG. 5 or the like, in order to allow the first jaw 1101 and the second jaw 1102 to pitch-rotate, the first wire 301 and the fifth wire 305, which are first jaw wires, are moved in the same direction and respectively wound around the ninth pulley 219 and the tenth pulley 220, which are operation part pitch main pulleys, and the second wire 302 and the sixth wire 306, which are second jaw wires, are moved in the same direction and respectively wound around the ninth pulley 219 and the tenth pulley 220 and the nineteenth pulley 229 and the twentieth pulley 230, which are operation part pitch main pulleys. In addition, as a rotating force is transmitted to the end tool 1100 through the power transmission part 300, the jaws 1103 of the end tool 1100 perform the pitch motion.

Since the pitch frame 208 is connected to the yaw frame 207, and the yaw frame 207 connects the handle 204, the first rotation shaft 241, the second rotation shaft 242, and the third rotation shaft 243 to each other, when the pitch frame 208 is rotated around the sixth rotation shaft 246, the yaw frame 207, the handle 204, the first rotation shaft 241, the second rotation shaft 242, and the third rotation shaft 243 connected to the pitch frame 208 may be rotated together with the pitch frame 208. That is, when the pitch operation part 201 is rotated around the sixth rotation shaft 246, the actuation operation part 203 and the yaw operation part 202 may be rotated together with the pitch operation part 201.

In summary, in the electrocautery surgical instrument 10 according to an embodiment of the present disclosure, the pulleys may be formed on respective joint points (an actuation joint, a yaw joint, and a pitch joint), the wires (the first jaw wires or the second jaw wires) may be wound around the pulleys, the rotational operations (actuation rotation, yaw rotation, and pitch rotation) of the operation part 200 may cause the movement of each wire, which in turn induces a desired motion of the end tool 1100. Furthermore, an auxiliary pulley may be formed at one side of each of the pulleys, and the wires may not be wound several times around one pulley due to the auxiliary pulley.

FIG. 5 is a view schematically illustrating only a configuration of pulleys and wires constituting joints of the electrocautery surgical instrument 10 according to an embodiment of the present disclosure illustrated in FIG. 1. In FIG. 5, the at least one relay pulley 235 that are not related to the operation of joints and are used to reroute the wires is omitted.

Referring to FIG. 5, the operation part 200 may include the first pulley 211, the second pulley 212, the third pulley 213, the fourth pulley 214, the fifth pulley 215, a sixth pulley 216, the seventh pulley 217, the eighth pulley 218, the ninth pulley 219, and the tenth pulley 220 that are related to a rotational motion of the first jaw 1101.

In addition, the operation part 200 may include the eleventh pulley 221, the twelfth pulley 222, the thirteenth pulley 223, the fourteenth pulley 224, the fifteenth pulley 225, a pulley 226, the seventeenth pulley 227, the eighteenth pulley 228, the nineteenth pulley 229, and the twentieth pulley 230, which are related to a rotational motion of the second jaw 1102. In addition, the operation part 200 may include the actuation pulley 262 associated with the rotational motion of the first jaw 1101 and the rotational motion of the second jaw 1102. (The arrangement and structure of each of the pulleys of the operation part 200 are the same in principle as the arrangement and structure of each of the pulleys of the end tool 1100, and thus specific designations of some reference numerals are omitted in the drawings).

The first pulley 211 and the second pulley 212 and the eleventh pulley 221 and the twelfth pulley 222 may be formed to be independently rotatable around the same shaft, which may be the second rotation shaft 242. Here, the first pulley 211 and the second pulley 212 may be formed as two pulleys facing each other and independently rotatable. Similarly, the eleventh pulley 221 and the twelfth pulley 222 may be formed as two pulleys facing each other and independently rotatable, and in this case, the two pulleys may have different diameters.

The third pulley 213 and the fourth pulley 214 and the thirteenth pulley 223 and the fourteenth pulley 224 may be formed to be independently rotatable around the same shaft, which may be the third rotation shaft 243. In this case, the third pulley 213 and the fourth pulley 214 and the thirteenth pulley 223 and the fourteenth pulley 224 may be formed as two pulleys facing each other and independently rotatable.

The fifth pulley 215 and the fifteenth pulley 225 may be formed to be rotatable independently of each other around the same shaft, which is the fourth rotation shaft 244.

The seventh pulley 217 and the eighth pulley 218 and the seventeenth pulley 227 and the eighth pulley 228 may be formed to be rotatable independently of each other around the same shaft, that is the fifth rotation shaft 245. In this case, the seventh pulley 217 and the eighth pulley 218 may be formed to have different diameters. In addition, the seventeenth pulley 227 and the eighteenth pulley 228 may be formed to have different diameters.

The ninth pulley 219 and the tenth pulley 220 and the nineteenth pulley 229 and the twentieth pulley 230 may be formed to be rotatable independently of each other around the same shaft, that is the sixth rotation shaft 246.

The first wire 301 is wound around the actuation pulley 262 after sequentially passing through the ninth pulley 219, the seventh pulley 217, the fifth pulley 215, the third pulley 213, and the first pulley 211 of the operation part 200, and then is coupled to the actuation pulley 262 by the fastening member 262a. In the other embodiment, the fifth wire 305 sequentially passes through the tenth pulley 220, the eighth pulley 218, the sixth pulley 216, the fourth pulley 214, and the second pulley 212 of the operation part 200 and is coupled to the actuation pulley 262 by the fastening member 262a. Thus, when the actuation pulley 262 is rotated, the first wire 301 and the fifth 305 are wound around or unwound from the actuation pulley 262, and accordingly, the first jaw 1101 is rotated.

The sixth wire 306 is wound around the actuation pulley 262 after sequentially passing through the nineteenth pulley 229, the seventeenth pulley 227, the fifteenth pulley 225, the thirteenth pulley 223, and the eleventh pulley 221 of the operation part 200, and then is coupled to the actuation pulley 262 by the fastening member 262a. In the other embodiment, the second wire 302 sequentially passes through the twentieth pulley 230, the eighteenth pulley 228, the sixteenth pulley 226, the fourteenth pulley 224, and the twelfth pulley 222 of the operation part 200 and is coupled to the actuation pulley 262 by the fastening member 262a. Thus, when the actuation pulley 262 is rotated, the second wire 302 and the sixth wire 306 are wound around or unwound from the actuation pulley 262, and accordingly, the second jaw 1102 is rotated.

### Conceptual Diagram of Pulleys and Wires

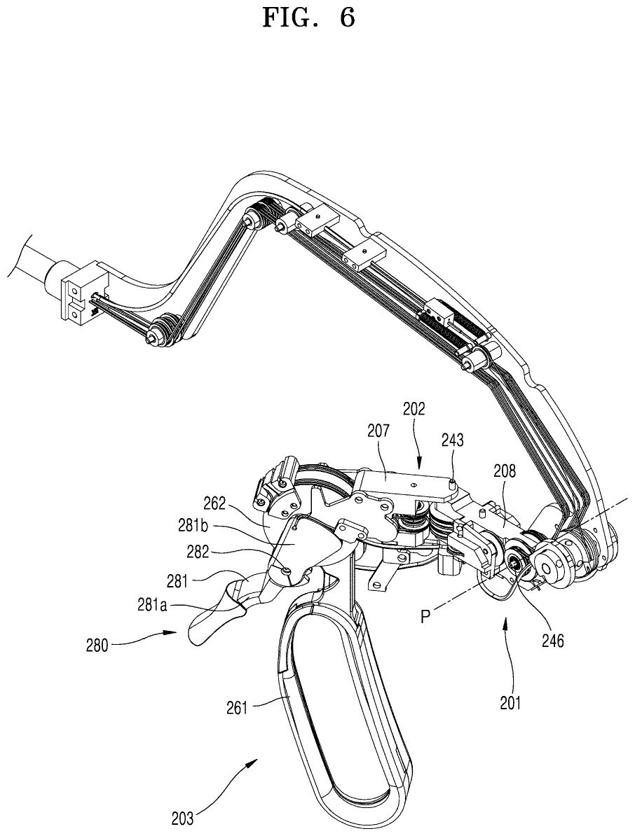

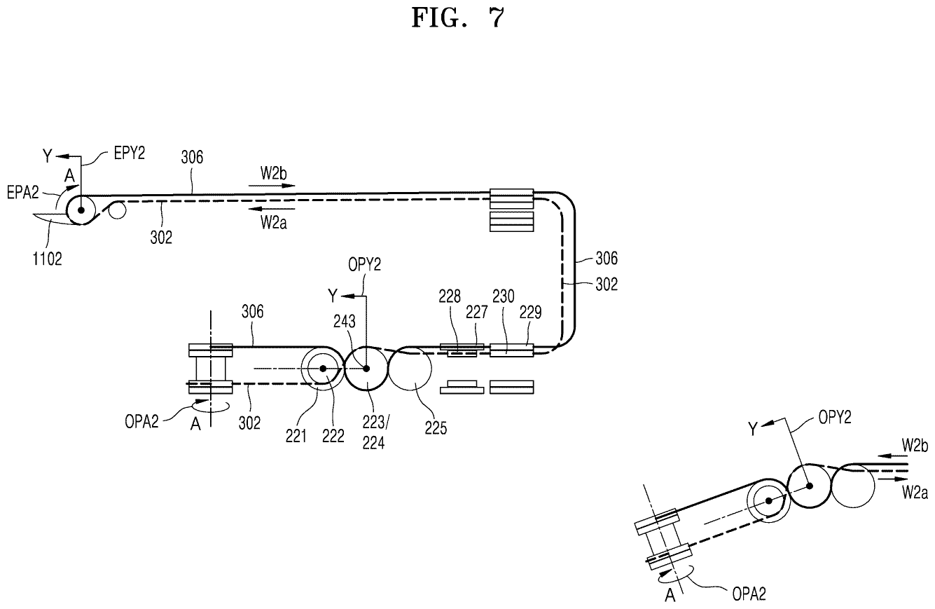

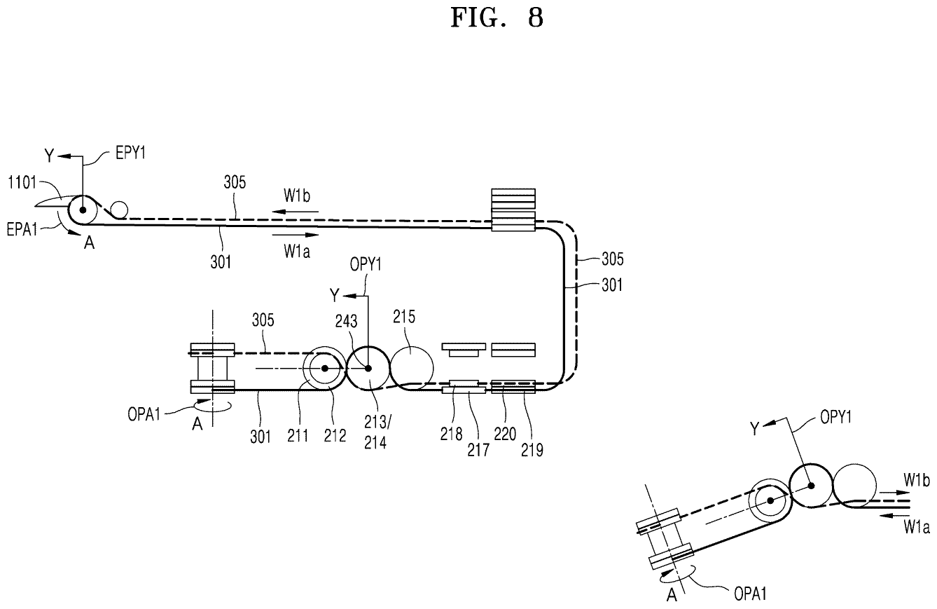

FIGS. 7 and 8 are diagrams illustrating a configuration of pulleys and wires, which are associated with an actuation motion and a yaw motion of the electrocautery surgical instrument 10 according to an embodiment of the present disclosure illustrated in FIG. 1, for each of the first jaw 1101 and the second jaw 1102, respectively. FIG. 7 is a diagram illustrating only pulleys and wires related to the second jaw 1102, and FIG. 8 is a diagram illustrating only pulleys and wires related to the first jaw 1101. In addition, FIG. 6 is a perspective view illustrating a yaw motion of the electrocautery surgical instrument of FIG. 1.

First, a wire operation in an actuation motion is described below.

Referring to FIG. 8, when the actuation lever 261 rotates around the first rotation shaft 241 in a direction of a first arrow OPA1, the actuation pulley 262 connected to the actuation lever 261 may be rotated, and the first wire 301 and the fifth wire 305 wound around the actuation pulley 262 may be moved in a first direction W1a and a second direction W1b, respectively, and as a result, the first jaw 1101 of the end tool 1100 may be rotated in a direction of a second arrow EPA1.

Referring to FIG. 7, when the actuation lever 261 rotates around the rotation shaft 241 in a direction of a third arrow OPA2, the actuation pulley 262 connected to the actuation lever 261 may be rotated, and thus both strands of the second wire 302 and the sixth wire 306 wound around the actuation pulley 262 may be moved in a third direction W2a and a fourth direction W2b, respectively, and as a result, the second jaw 1102 of the end tool 1100 may be rotated in a direction of a fourth arrow EPA2. Thus, when the user operates the actuation lever 261 in a direction closer to the handle 204, a motion in which the first jaw 1101 and the second jaw 1102 move closer to each other is performed.

Next, a wire operation in the yaw motion will be described.

First, since the third rotation shaft 243 is connected to the first rotation shaft 241 and the second rotation shaft 242 by the yaw frame (see 207 of FIG. 3), the third rotation shaft 243 and the first rotation shaft 241 and the second rotation shaft 242 may be integrally rotated together.

Referring to FIG. 8, when the handle 204 rotates around the third rotation shaft 243 in the direction of a fifth arrow OPY1, the actuation pulley 262, the first pulley 211, the second pulley 212, the third pulley 213, and the fourth pulley 214 and the first wire 301 and the fifth wire 305 wound therearound may be rotated as a whole around the third rotation shaft 243, and as a result, the first wire 301 and the fifth wire 305 wound around the third pulley 213 and the fourth pulley 214 may be moved in the first direction W1a and the second direction W1b, respectively, which causes the first jaw 1101 of the end tool 1100 to rotate in aa direction of a sixth arrow EPY1.

Referring to FIG. 7, when the handle 204 rotates around the third rotation shaft 243 in a direction of a seventh arrow OPY2, the actuation pulley 262, the eleventh pulley 221, the twelfth pulley 222, the thirteenth pulley 223, and the fourteenth pulley 224 and the second wire 302 and the sixth wire 306 wound therearound may be rotated as a whole around the third rotation shaft 243, and as a result, the second wire 302 and the sixth wire 306 wound around the thirteenth pulley 223 and the fourteenth pulley 224 may be moved in directions opposite to the third direction W2a and the fourth direction W2b, respectively, which causes the second jaw 1102 of the end tool 1100 to rotate in the direction of an eighth arrow EPY2.

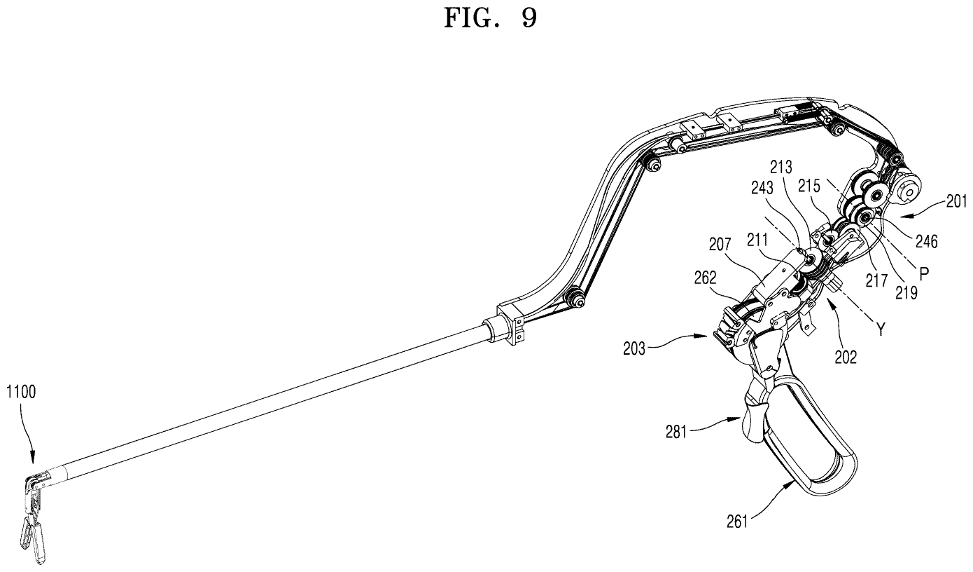

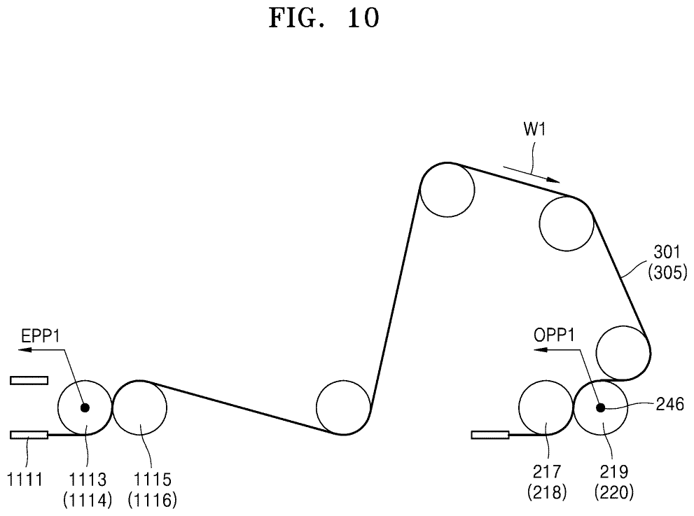

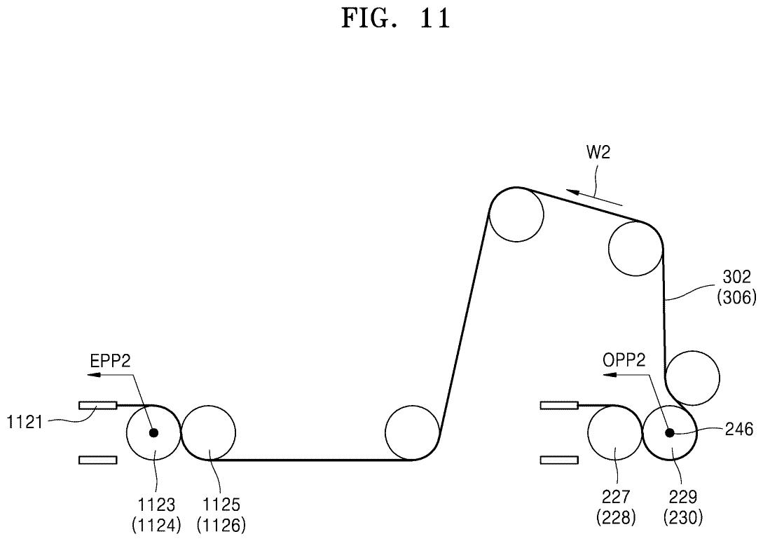

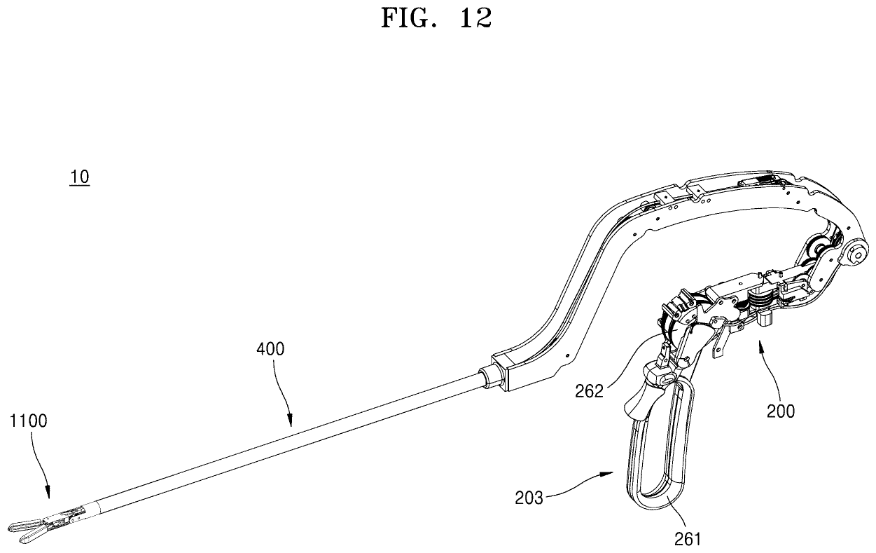

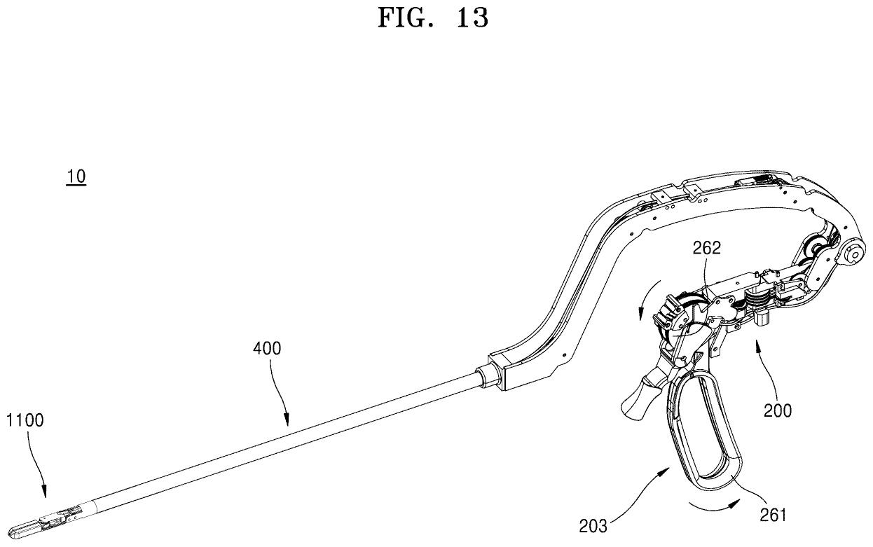

FIGS. 10 and 11 are diagrams illustrating a configuration of the pulleys and the wires, which are associated with the pitch motion of the electrocautery surgical instrument 10 according to an embodiment of the present disclosure illustrated in FIG. 1, for each of the first jaw 1101 and the second jaw 1102, respectively. FIG. 11 is a diagram illustrating only the pulleys and the wires related to the second jaw 1102, and FIG. 10 is a diagram illustrating only the pulleys and the wires related to the first jaw 1101. As shown in FIG. 7 and elsewhere herein, there may be two pulleys related to the pitch motion, and both strands of each wire may be wound in the same path, which is illustrated with a single line in FIG. 10. In addition, FIG. 9 is a perspective view illustrating the pitch motion of the electrocautery surgical instrument of FIG. 1.

Referring to FIG. 10, when the handle 204 is rotated around the sixth rotation shaft 246 in the direction of a ninth arrow OPP1, the actuation pulley 262, the seventh pulley 217, the ninth pulley 219, and the like, and the first wire 301 and the like wound therearound may be rotated as a whole around the sixth rotation shaft 246. Since the first wire 301 and the fifth wire 305, which are the first jaw wires, are wound around upper portions of the ninth pulley 219 and the tenth pulley 220, the first wire 301 and the fifth wire 305 may be moved in a direction of a tenth arrow W1. As a result, the first jaw 1101 of the end tool 1100 may rotate in the direction of an eleventh arrow EPP1.

Referring to FIG. 11, when the handle 204 is rotated around the sixth rotation shaft 246 in the direction of a twelfth arrow OPP2, the actuation pulley 262, the seventeenth pulley 227, the nineteenth pulley 229, and the like, and the second wire 302 and the like wound therearound may be rotated as a whole around the sixth rotation shaft 246. Since the second wire 302 and the sixth wire 306, which are the second jaw wires, are wound around lower portions of the nineteenth pulley 229 and the twentieth pulley 230, the second wire 302 and the sixth wire 306 may be moved in the direction of a thirteenth arrow W2. As a result, the second jaw 1102 of the end tool 1100 may rotate in the direction of a fourteenth arrow EPP2.

Thus, the actuation, yaw, and pitch operations can be operated independently of one another.

### Actuation Lever Operation

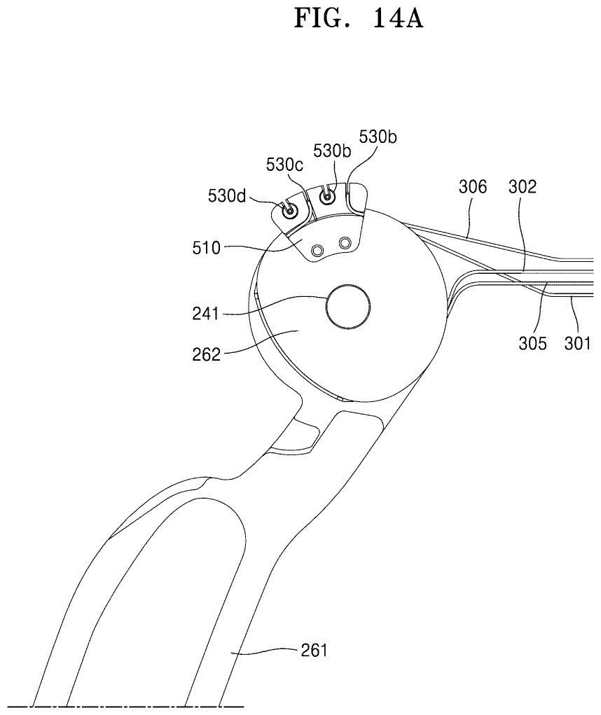

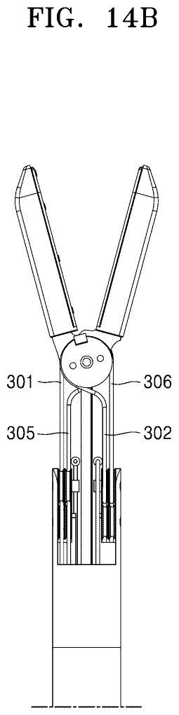

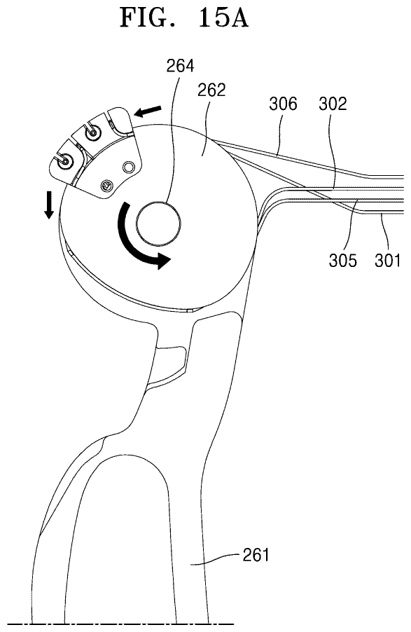

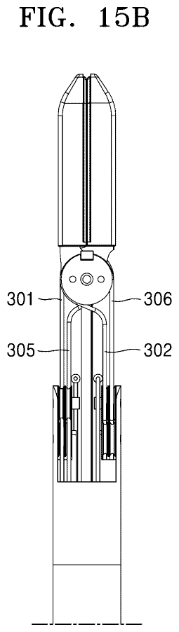

FIGS. 12 and 13 are perspective views illustrating an operation of the actuation lever 261 of the electrocautery surgical instrument 10 illustrated in FIG. 1, and FIGS. 14A to 15B are diagrams illustrating the movement of the wires during the operation of the actuation lever 261 of the electrocautery surgical instrument 10 illustrated in FIG. 1.

FIG. 12 shows the electrocautery surgical instrument 10 of FIG. 1 with a case removed, illustrating the actuation lever 261 in a non-operating state, and FIG. 13 shows the electrocautery surgical instrument 10 illustrated in FIG. 1 with the case removed, illustrating the actuation lever 261 in an operating state.

Referring to FIGS. 12 and 13, in the electrocautery surgical instrument 10 according to an embodiment of the present disclosure, the actuation pulley 262 can be rotated by gripping the handle 204 with the palm and pulling the actuation lever 261 toward the handle 204 while inserting a finger into the actuation lever 261. That is, an actuation motion can be performed by operating one lever.

FIG. 14A is a side view illustrating the wires disposed at the actuation pulley 262 of the electrocautery surgical instrument 10 illustrated in FIG. 1, and FIG. 14B is a plan view illustrating the wires disposed at the end tool 1100. FIG. 15A is a side view illustrating the movement of the wires at the actuation pulley 262 during the actuation lever operation, and FIG. 15B is a plan view illustrating the movement of the wires at the end tool 1100.

Referring to FIGS. 5 and 14A to 15B, in the electrocautery surgical instrument 10 according to an embodiment of the present disclosure, the first wire 301 and the fifth wire 305, which are the first jaw wires, and the second wire 302 and the sixth wire 306, which are the second jaw wires, may all be coupled to a single actuation pulley 262, and thus, by appropriately positioning each wire, the movement of each wire can may be varied solely by the rotation of a single pulley. That is, by rotating the actuation pulley 262 in any one direction through the actuation lever 261, the first jaw 1101 and the second jaw 1102 may rotate in opposite directions. In other words, depending on the rotation of the actuation pulley 262, the first jaw 1101 and the second jaw 1102 may perform opening and closing motions, allowing them to open or close.

Specifically, the first wire 301 and the fifth wire 305, which are the first jaw wires, may be wound in opposite directions around the actuation pulley 262. For example, as shown in FIG. 5, the first wire 301 may be wound in the counterclockwise direction and the fifth wire 305 may be wound in the clockwise direction. Similarly, the second wire 302 and the sixth wire 306, which are the second jaw wires, may be wound in opposite directions around the actuation pulley 262. For example, as shown in FIG. 5, the second wire 302 may be wound in the clockwise direction and the sixth wire 306 may be wound in the counterclockwise direction.

At this point, when the actuation pulley 262 rotates in the counterclockwise direction, the first wire 301 may be wound around the actuation pulley 262 and the fifth wire 305 may be unwound from the actuation pulley 262. Accordingly, at the first jaw pulley 1111 of the end tool 1100, the first wire 301 may be unwound, and the fifth wire 305 may be wound, causing the first jaw pulley 1111 of the end tool 1100 to rotate in the counterclockwise direction.

In addition, when the actuation pulley 262 rotates in the counterclockwise direction, the sixth wire 306 may be wound around the actuation pulley 262 and the second wire 302 may be unwound from the actuation pulley 262. Accordingly, at the second jaw pulley 1121 of the end tool 1100, the wire 306 is unwound, and the second wire 302 may be wound, causing the second jaw pulley 1121 of the end tool to rotate in the clockwise direction.

Similarly, when the actuation pulley 262 rotates in the clockwise direction, the first jaw pulley 1111 may be rotated in the clockwise direction and the second jaw pulley 1121 may be rotated in the counterclockwise direction.

Accordingly, when the actuation pulley 262 rotates, the first jaw pulley 1111 and the second jaw pulley 1121 may be rotated in opposite directions, causing the first jaw 1101 and the second jaw 1102 of the end tool 1100 to open or close.

In the other embodiment, as described above, when the actuation lever 261 is rotated in a direction closer to the handle 204, the actuation pulley 262 may rotate along with the actuation lever 261, thereby allowing the actuation motion of the end tool 1100 to be performed. A rotational force of the actuation lever 261 may increase as a rotation angle of the actuation lever 261 increases, and a tension of the wire positioned on the actuation pulley 262 may also increase. That is, when the actuation lever 261 and the actuation pulley 262 rotate integrally, the rotation angle of the actuation lever 261 may be proportional to the tension of the wire positioned on the actuation pulley 262.

When the tension of the wire continues to increase as the rotation angle of the actuation lever 261 increases, excessive tension may be applied to the wire, which may result in the wire being permanently stretched or excessive internal force being exerted on the instrument.

The present disclosure may prevent excessive actuation force from being applied to the end tool 1100 by arranging an elastic member 266 between the actuation lever 261 and the actuation pulley 262 to adjust the rotational force transmitted to the actuation pulley 262. Hereinafter, the operations of the actuation pulley 262 and the elastic member 266 according to the operation of the actuation lever 261 will be described.

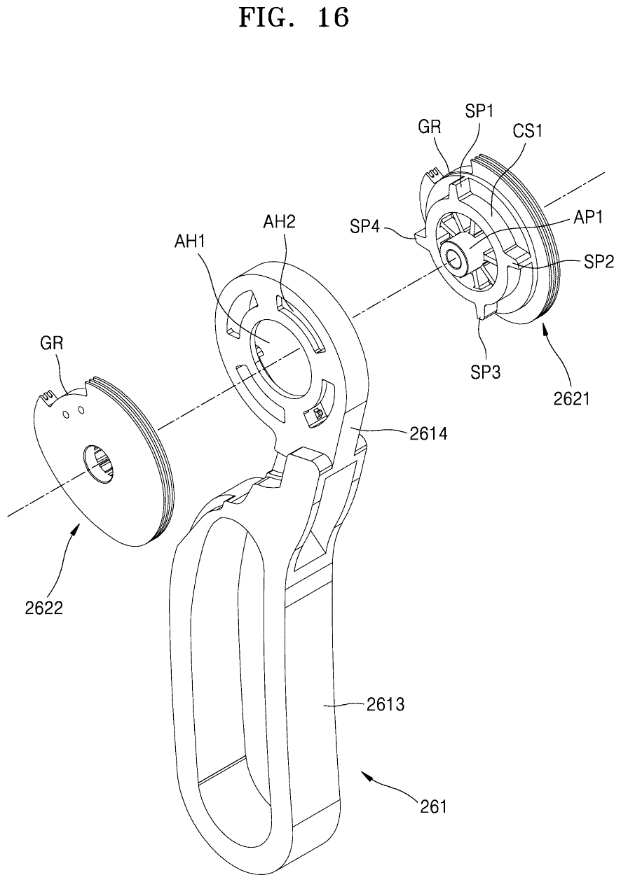

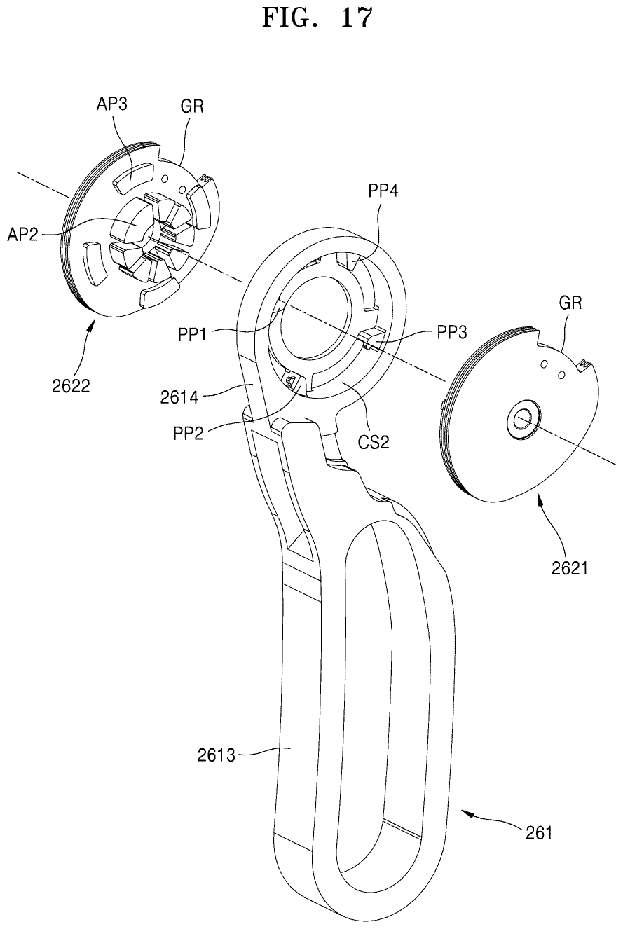

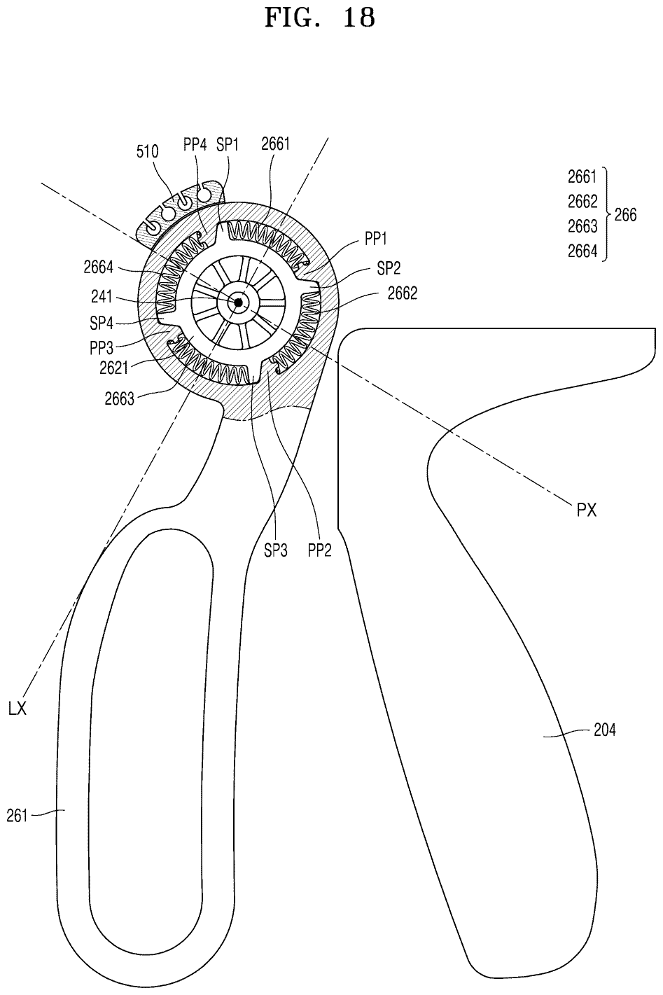

FIGS. 16 and 17 are exploded perspective views of the actuation lever 261 and the actuation pulley 262 of the electrocautery surgical instrument 10 of FIG. 1. FIGS. 18 to 20 are views illustrating an operation process of the actuation lever 261 of the electrocautery surgical instrument 10 of FIG. 1.

Referring to FIGS. 16 to 20, the actuation lever 261 and the actuation pulley 262 may be coupled to each other. The actuation lever 261 and the actuation pulley 262 may be coupled to each other to rotate integrally as a unit or to rotate relative to each other.

The actuation lever 261 may include a first lever part 2613 and a second lever part 2614 formed to extend from the first lever part 2613. The actuation lever 261 may be rotated by a user's operation to rotate the actuation pulley 262.

The first lever part 2613 is defined as a region of the actuation lever 261 through which a user can insert a finger. The first lever part 2613 may be formed in the shape of a finger ring, and the user may rotate the actuation lever 261 while inserting a finger into the first lever part 2613.

The second lever part 2614 is defined as a region that is coupled to the actuation pulley 262. The second lever part 2614 may be formed in various shapes that can be assembled with the actuation pulley 262. For example, as shown in FIGS. 16 and 17, the second lever part 2614 may include at least one assembly hole AH. The second lever part 2614 may include a first assembly hole AHI at the center thereof and a plurality of second assembly holes AH2 arranged to surround the first assembly hole AH1. As assembly protrusions AP of the actuation pulley 262, which will be described later, may be fitted into the second assembly hole AH2, the actuation pulley 262 may be stably coupled to the actuation lever 261.

The elastic member 266 may be seated on the second lever part 2614. The second lever part 2614 may include a second seating surface CS2 on which a first pulley body 2621 is seated, and the elastic member 266 may be disposed on the second seating surface CS2 between the first pulley body 2621 and the second lever part 2614.

The second lever part 2614 may include a pressing protrusion PP. The pressing protrusion PP may be formed to protrude toward a central portion of the second lever part 2614 on the second seating surface CS2. That is, based on a coupling state of the actuation lever 261 and the actuation pulley 262, the pressing protrusion PP may be formed to protrude toward the actuation pulley 262.

The pressing protrusion PP may support one side of the elastic member 266 disposed on the second seating surface CS2. That is, the pressing protrusion PP may support the elastic member 266, and may receive a certain reaction force from the elastic member 266. In addition, with the rotation of the actuation lever 261, the pressing protrusion PP may apply a rotational force to the elastic member 266.

The pressing protrusion PP may include a plurality of pressing protrusions. When a single elastic member 266 is disposed on the second lever part 2614, the second lever part 2614 may have one pressing protrusion PP. On the other hand, when a plurality of elastic members 266 are disposed on the second lever part 2614, the second lever part 2614 may be provided with the pressing protrusions PP in a number corresponding to the number of the elastic members 266. That is, even when the plurality of pressing protrusions are disposed, each one of the pressing protrusions may support one elastic member 266.

The actuation pulley 262 may have at least one wire positioned thereon. As described above, the first wire 301, the second wire 302, the fifth wire 305, and the sixth 306 for the actuation motion of the end tool 1100 may be positioned on the actuation pulley 262. In addition, the wire fastening member 510 to which a wire is fastened may be coupled to one side of the actuation pulley 262, and the wire fastened to the wire fastening member 510 may be stably positioned along an outer circumferential surface of the actuation pulley 262.

The actuation pulley 262 may have the assembly protrusions AP. The assembly protrusions AP may be fitted into the second assembly hole AH2 of the actuation lever 261, thereby allowing the actuation pulley 262 to be stably coupled to the actuation lever 261. However, the present disclosure is not limited thereto, and the actuation pulley 262 may be formed in various shapes that can be assembled with the actuation lever 261.

The actuation pulley 262 may be formed as a single member and coupled to the actuation lever 261, or as a plurality of members, each assembled to the actuation lever 261. In the other embodiment, the following description will focus on an embodiment in which the actuation pulley 262 includes the first pulley body 2621 and a second pulley body 2622. The first pulley body 2621 and the second pulley body 2622 may, respectively, be assembled to one surface and another surface of the actuation lever 261.

The first pulley body 2621 may include an engagement groove GR. The engagement groove GR may be formed on one side of the first pulley body 2621, and the wire fastening member 510 may be seated in the engagement groove GR. Accordingly, the wire fastening member 510 and the actuation pulley 262 may rotate together to push or pull the wire.

The first pulley body 2621 may include a first assembly protrusion AP1. The first assembly protrusion AP1 may protrude from the one side of the first pulley body 2621, and may be inserted into the first assembly hole AH1 of the actuation lever 261. The first assembly protrusion AP1 may be formed as a single member as shown in FIG. 16, or may be formed as a plurality of protrusions.

The first pulley body 2621 may include a first seating surface CS1 on which the elastic member 266 may be seated, and the elastic member 266 may be elastically deformably disposed between the second seating surface CS2 of the second lever part 2614 and the first seating surface CS1.

The first pulley body 2621 may include a support protrusion SP. Based on the state in which the actuation lever 261 is coupled to the actuation pulley 262, the support protrusion SP may be formed to protrude from the first seating surface CS1 toward the second seating surface CS2.

The support protrusion SP may support another side of the elastic member 266 disposed between the first seating surface CS1 and the second seating surface CS2. That is, the support protrusion SP supports the elastic member 266, and may receive a certain reaction force from the elastic member 266.

The second pulley body 2622 may include an engagement groove GR. Similar to the first pulley body 2621, the engagement groove GR is formed on one side of the second pulley body 2622, allowing the wire fastening member 510 to be seated in the engagement groove GR. Accordingly, the wire fastening member 510 and the actuation pulley 262 may rotate together to push or pull the wire.

The second pulley body 2622 may include a second assembly protrusion AP2 and third assembly protrusions AP3.

The second assembly protrusion AP2 protrudes from a central region of the second pulley body 2622, and may be inserted into the first assembly hole AHI of the actuation lever 261. In addition, the first assembly protrusion AP1 may be engaged with the second assembly protrusion AP2. For example, the second assembly protrusion AP2 may be formed as a single ring-shaped protrusion or as a plurality of protrusions arranged radially. As described above, the second assembly protrusion AP2 may be formed to allow the insertion of the first assembly protrusion AP1 at its center, thereby improving the coupling stability between the actuation lever 261, the first pulley body 2621, and the second pulley body 2622.

The third assembly protrusion AP3 may be arranged to surround the second assembly protrusion AP2 and may be inserted into the second assembly holes AH2 of the actuation lever 261, respectively. To enable the actuation pulley 262 to rotate relative to the actuation lever 261, a width of the third assembly protrusion AP3 may be configured smaller than a width of the second assembly hole AH2.

The elastic member 266 may be disposed between the actuation lever 261 and the actuation pulley 262. The elastic member 266 transmits at least a portion of the rotational force received from the actuation lever 261 to the actuation pulley 262, thereby allowing the actuation pulley 262 to rotate in response to the rotation of the actuation lever 261.

The elastic member 266 may include various members that are elastically deformable. For example, the elastic member 266 may be a compression spring, and in this case, an elastic modulus or the like of the compression spring may be variously selected.

The elastic member 266 may be supported on one side by the pressing protrusion PP of the actuation lever 261, and on another side by the actuation pulley 262, specifically, the support protrusion SP of the first pulley body 2621. In this case, the elastic member 266 may be disposed in a certain compressed state or formed as a member with a sufficient elastic modulus, and thus may be disposed with a restoring force between the pressing protrusion PP and the support protrusion SP. Accordingly, the elastic member 266 may transmit the rotational force received from the pressing protrusion PP to the support protrusion SP, and depending on the magnitude of the rotational force, the actuation pulley 262 may rotate relative to the actuation lever 261 when the actuation lever 261 rotates.

One or more elastic members 266 may include a plurality of elastic members. When the elastic member 266 includes one elastic member, the pressing protrusion PP and support protrusion SP also includes one pressing protrusion and one support protrusion, respectively. On the other hand, when the elastic member 266 includes the plurality of elastic members, the number of the pressing protrusion PP and the number of the support protrusion SP may each correspond to the number of the plurality of elastic members 266. Since the elastic member 266 is supported on opposite sides by the pressing protrusion PP and the support protrusion SP, the elastic member 266 may be disposed with the restoring force between the pressing protrusion PP and the support protrusion SP. When the actuation operation part 203 includes the plurality of elastic members, the restoring forces of the plurality of elastic members 266 may be distributed, thereby distributing the force applied to the pressing protrusion PP and the support protrusion SP. Accordingly, the actuation operation part 203 may be driven with a stable structure.

In the other embodiment in which the actuation operation part 203 includes the plurality of elastic members 266, the plurality of elastic members 266 may be disposed to be spaced apart at preset intervals between the actuation lever 261 and the actuation pulley 262. In addition, in response thereto, the plurality of pressing protrusions PP and a plurality of support protrusions SP may also be disposed at regular intervals.

In an optional embodiment, the plurality of elastic members 266 may be disposed between the actuation lever 261 and the actuation pulley 262 to be symmetrically spaced apart from each other around the first rotation shaft 241. In response thereto, each of the plurality of pressing protrusions PP and the plurality of support protrusions SP may also be disposed at regular intervals.

With the plurality of elastic members 266 disposed to be spaced apart at regular intervals as described above, the restoring forces of the elastic member 266 may be effectively distributed, thereby enhancing the structural stability of the actuation operation part 203.

In the present disclosure, the shape, material, or the like of the elastic member 266 may vary. The number of the elastic member 266 provided in the actuation operation part 203 may also be varied. In the present disclosure, the rotational force transmitted from the actuation lever 261 to the actuation pulley 262 may be controlled by appropriately selecting the specifications and the number of the elastic member 266.

Specifically, the elastic member 266 may be assembled in an elastically deformed state between the actuation lever 261 and the actuation pulley 262. That is, the elastic member 266 is disposed with a certain restoring force due to its elastic deformation, and this restoring force may be transmitted to the actuation lever 261 and the actuation pulley 262 through the pressing protrusion PP and the support protrusion SP, respectively.

When a certain external force is applied to the elastic member 266 as the actuation lever 261 rotates, the operation of the elastic member 266 may vary depending on the magnitude of the external force. When the external force applied to the elastic member 266 is less than the restoring force of the elastic member 266, no additional deformation occurs in the elastic member 266 because the external force is less than the force required to further deform the elastic member 266. On the other hand, when the external force applied to the elastic member 266 is greater than the restoring force of the elastic member 266, the external force may further deform the elastic member 266.

The restoring force of the elastic member 266 is proportional to the amount of elastic deformation of the elastic member 266. Accordingly, when the elastic member 266 is further deformed by the external force, the restoring force of the elastic member 266 may increase accordingly. In this process, as a portion of the external force applied to the elastic member 266 is distributed, the restoring force of the elastic member 266 may function as a threshold value capable of adjusting rotational forces above a certain magnitude. Such a threshold value may vary depending on the material, shape, and the like of the elastic member 266. In addition, the actuation operation part 203 may be set to have an appropriate threshold value by adjusting the sum of the restoring forces of the elastic members 266 through the inclusion of the plurality of elastic members 266.

Hereinafter, the operation of the actuation lever 261 will be described, focusing on an embodiment in which the actuation operation part 203 of the electrocautery surgical instrument 10 of the present disclosure includes four elastic members, that is, a first elastic member 2661, a second elastic member 2662, a third elastic member 2663, and a fourth elastic member 2664. Accordingly, the actuation lever 261 may include a first pressing protrusion PP1, a second pressing protrusion PP2, a third pressing protrusion PP3, and a fourth pressing protrusion PP4, and the actuation pulley 262 may include a first support protrusion SP1, a second support protrusion SP2, a third support protrusion SP3, and a fourth support protrusion SP4.

Next, operations of the elastic member 266 and the actuation pulley 262 in response to a rotation of the actuation lever 261 will be described with reference to FIGS. 18 to 20.

First, regarding the rotation of the actuation lever 261, in an initial state, a tangent line from the first rotation shaft 241, which is in contact with one side of the actuation lever 261, may be defined as a lever reference axis LX. In addition, in a rotated state, a tangent line from the first rotation shaft 241, which is in contact with the one side of the actuation lever 261, is defined as a lever line LL. Accordingly, an angle between the lever reference axis LX and the lever line LL corresponds to the rotation angle of the actuation lever 261.

Similarly, in the present disclosure, regarding the rotation of the actuation pulley 262, in an initial state, a tangent line from the first rotation shaft 241, which is in contact with one side of the wire fastening member 510, is defined as a pulley reference axis PX. In addition, in a rotated state, a tangent line from the first rotation shaft 241, which is in contact with the one side of the wire fastening member 510, is defined as a pulley line PL. Accordingly, an angle between the pulley reference axis PX and the pulley line PL corresponds to the rotation angle of the actuation pulley 262. FIG. 18 illustrates the initial state before the actuation lever 261 rotates toward the handle 204.

In the initial state, the first elastic member 2661 is supported on one side by the first pressing protrusion PP1 and on another side by the first support protrusion SP1. The second elastic member 2662 is supported on one side by the second pressing protrusion PP2 and on another side by the second support protrusion SP2, and similarly, the third elastic member 2663 and the fourth elastic member 2664 are supported by the third pressing protrusion PP3 and the third support protrusion SP3, and the fourth pressing protrusion PP4 and the fourth support protrusion SP4, respectively.

Since each elastic member 266 is elastically deformed and disposed with a certain restoring force, a certain force is applied to the pressing protrusion PP and the support protrusion SP supporting opposite sides of the elastic member 266. Accordingly, in the initial state, the first pressing protrusion PPI may be disposed to be in contact with the second support protrusion SP2. Similarly, the second pressing protrusion PP2 is disposed to be in contact with the third support protrusion SP3, the third pressing protrusion PP3 is disposed to be in contact the fourth support protrusion SP4, and the fourth pressing protrusion PP4 is disposed to be in contact the first support protrusion SP1. As described above, in the initial state, the actuation lever 261 and the actuation pulley 262 are coupled to each other such that the pressing protrusion PP comes into contact with the adjacent support protrusion SP.

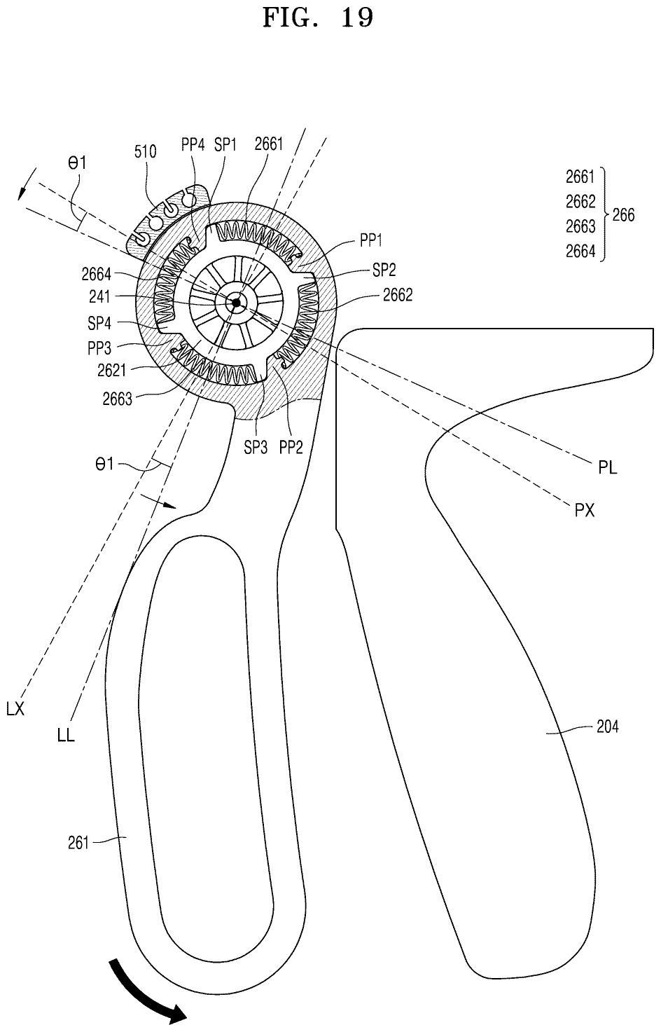

FIG. 19 illustrates a state in which the actuation lever 261 is rotated toward the handle 204 by a first rotation angle θ1.

When the actuation lever 261 is rotated toward the handle 204, the first pressing protrusion PP1 transmits the rotational force to the first elastic member 2661. When the actuation lever 261 is rotated by the first rotation angle θ1, a first rotational force is generated. When the first rotational force is less than a threshold value, the first pressing protrusion PP1 rotates while remaining in contact with the second support protrusion SP2 due to the restoring force of the first elastic member 2661.

That is, when the rotational force applied by the pressing protrusion PP to the elastic member 266 during the rotation of the actuation lever 261 is less than the threshold value, the elastic member 266 does not undergo elastic deformation but maintains its restoring force while rotating together with the actuation lever 261. Accordingly, the elastic member 266 transmits the entire first rotational force received from the pressing protrusion PP to the support protrusion SP, and as a result, the actuation pulley 262 may be rotated by the same first rotation angle θ1 as the actuation lever 261. Accordingly, the entire first rotational force is transmitted to the wire positioned on the actuation pulley 262, allowing tension corresponding to the first rotational force to be applied to the wire.

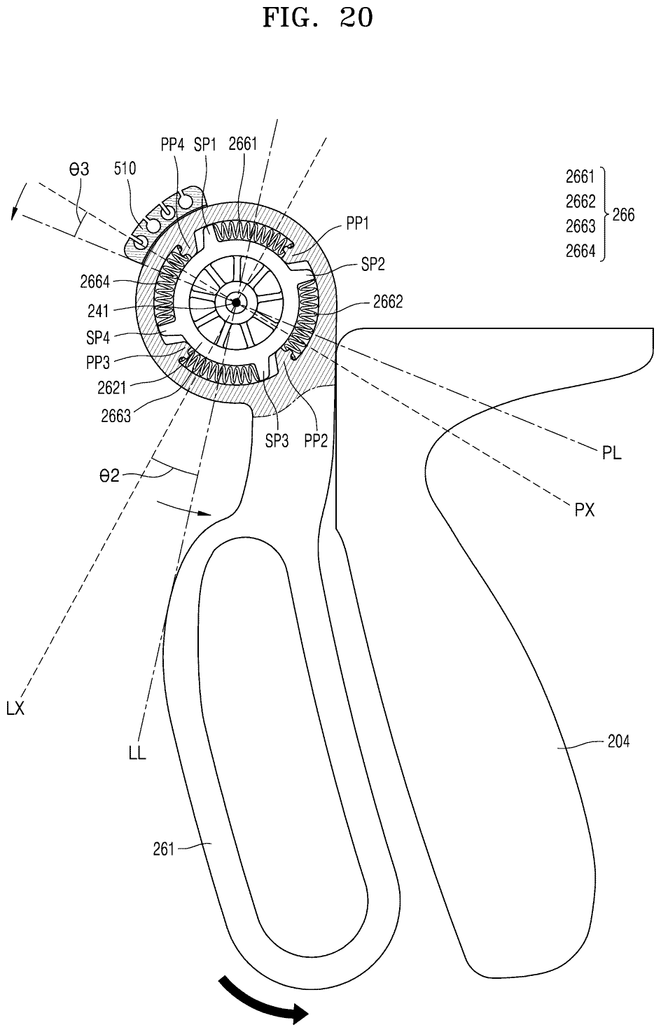

FIG. 20 illustrates a state in which the actuation lever 261 is rotated toward the handle 204 by a second rotation angle θ2.

When the actuation lever 261 is rotated by the second rotation angle θ2, a second rotational force is generated. When the second rotational force is greater than the threshold value, the first elastic member 2661 may be elastically deformed. When the first pressing protrusion PPI applies the second rotational force to the first elastic member 2661, the first elastic member 2661 is elastically compressed by a portion of the second rotational force and transmits the remaining portion of the second rotational force to the first support protrusion SP1. Since the first elastic member 2661 is compressed, the first pressing protrusion PP1 may have a certain gap from the second support protrusion SP2.

That is, when the rotational force applied by the pressing protrusion PP to the elastic member 266 during the rotation of the actuation lever 261 is greater than the threshold value, the elastic member 266 is elastically compressed by a portion of the rotational force and rotates. The elastic member 266 transmits the remaining portion of the rotational force to the support protrusion SP, so that the actuation pulley 262 is rotated by a third rotation angle θ3 that is less than the second rotation angle θ2 of the actuation lever 261. Accordingly, only the remaining portion of the second rotational force is transmitted to the wire positioned on the actuation pulley 262, which may reduce the tension acting on the wire. Accordingly, excessive tension on the wire can be prevented, thereby preventing a mechanical damage to the instrument.

In the electrocautery surgical instrument 10 according to an embodiment of the present disclosure, the actuation pulley 262 can rotate relative to the actuation lever 261 by arranging the elastic member 266 between the actuation lever 261 and the actuation pulley 262. That is, the elastic member 266 can adjust the rotational force transmitted from the actuation lever 261 to the actuation pulley 262. Accordingly, excessive tension is prevented from being applied to the wire connected to the actuation pulley 262, and the stability of the power transmission structure of the instrument can be improved.

An electrocautery surgical instrument according to the present disclosure incorporates a structure in which an actuation lever and an actuation pulley can rotate relative to each other through an elastic member, thereby preventing excessive tension from being applied to a wire connected to the actuation pulley. Accordingly, the stability of an end tool and its power transmission structure can be improved, while preventing mechanical damage to the instrument.

The present disclosure has been described above in relation to its preferred embodiments. It will be understood by those skilled in the art that various changes in form and details may be made therein without departing from the essential features of the present disclosure. Therefore, the disclosed embodiments should only be considered in a descriptive sense and not for purposes of limitation. The scope of the present disclosure is defined not by the detailed description of the disclosure but by the appended claims, and all differences within the scope will be construed as being included in the present disclosure.

What is claimed is:

1. An operation part of a surgical instrument equipped with an end tool, the operation part comprising:
a handle; and
an actuation operation part disposed on one side of the handle and configured to control an actuation motion of the end tool,
wherein the actuation operation part includes:
an actuation lever rotatable around an actuation rotation shaft;
an actuation pulley having one or more wires positioned thereon coupled to the actuation lever, and rotatable in response to a rotation of the actuation lever; and
an elastic member elastically deformably disposed between the actuation lever and the actuation pulley, and configured to transmit at least a portion of a rotational force of the actuation lever to the actuation pulley.

2. The operation part of claim 1, wherein the elastic member is supported at one end by a pressing protrusion of the actuation lever and at another end by a support protrusion of the actuation pulley, and disposed with a predetermined restoring force between the pressing protrusion and the support protrusion.

3. The operation part of claim 2, wherein the elastic member is configured to rotate in correspondence with the actuation lever and transmit the rotational force to the actuation pulley.

4. The operation part of claim 2, wherein the elastic member is configured to elastically compress by a portion of a rotational force applied by the pressing protrusion and transmit a remaining portion of the rotational force to the actuation pulley when the rotational force is greater than a threshold value.

5. The operation part of claim 2, wherein the elastic member is configured to adjust, based on the predetermined restoring force, a rotational force received from the pressing protrusion when the actuation lever rotates.

6. The operation part of claim 5, wherein the elastic member is configured to elastically deform when the rotational force applied by the pressing protrusion when the actuation lever rotates is greater than the predetermined restoring force.

7. The operation part of claim 1, wherein
the actuation lever includes a pressing protrusion protruding toward the actuation pulley,
the actuation pulley includes a support protrusion protruding toward the actuation lever, and
the elastic member is disposed between the pressing protrusion and the support protrusion.

8. The operation part of claim 7, wherein the actuation lever includes a plurality of pressing protrusions,
wherein the plurality of pressing protrusions are disposed spaced apart at preset intervals.

9. The operation part of claim 1, wherein the actuation pulley is configured to receive at least a portion of the rotational force of the actuation lever from the elastic member and transmit the at least a portion of the rotational force to the one or more wires when the actuation lever rotates.

10. The operation part of claim 9, wherein the actuation pulley is configured to rotate in correspondence with the actuation lever and transmit the rotational force to the one or more wires.

11. The operation part of claim 9, wherein the actuation pulley is configured to receive a portion of the rotational force and transmit the portion of the rotational force to the one or more wires when the rotational force of the actuation lever is greater than a threshold value.

12. An electrocautery surgical instrument comprising:
an end tool rotatable in at least one direction;
an operation part including a handle and an actuation operation part disposed on one side of the handle and configured to control an actuation motion of the end tool; and
a power transmission part including an actuation wire connecting the end tool to the actuation operation part, and configured to transmit power from the actuation operation part to the end tool,
wherein the actuation operation part includes:
an actuation lever rotatable around an actuation rotation shaft;
an actuation pulley having the actuation wire positioned thereon coupled to the actuation lever, and rotatable in response to a rotation of the actuation lever; and
an elastic member elastically deformably disposed between the actuation lever and the actuation pulley, and configured to transmit at least a portion of a rotational force of the actuation lever to the actuation pulley.

13. The electrocautery surgical instrument of claim 12, wherein the elastic member is supported at one end by a pressing protrusion of the actuation lever and at another end by a support protrusion of the actuation pulley, and disposed with a predetermined restoring force between the pressing protrusion and the support protrusion.

14. The electrocautery surgical instrument of claim 13, wherein the elastic member is configured to rotate in correspondence with the actuation lever and transmit the rotational force to the actuation pulley.

15. The electrocautery surgical instrument of claim 13, wherein the elastic member is configured to elastically compress by a portion of a rotational force applied by the pressing protrusion and transmit a remaining portion of the rotational force to the actuation pulley when the rotational force is greater than a threshold value.

16. The electrocautery surgical instrument of claim 13, wherein the elastic member is configured to adjust, based on the predetermined restoring force, a rotational force received from the pressing protrusion when the actuation lever rotates.

17. The electrocautery surgical instrument of claim 16, wherein the elastic member is configured to elastically deform when the rotational force applied by the pressing protrusion when the actuation lever rotates is greater than the predetermined restoring force.

18. The electrocautery surgical instrument of claim 12, wherein the actuation lever includes a pressing protrusion protruding toward the actuation pulley, the actuation pulley includes a support protrusion protruding toward the actuation lever, and the elastic member is disposed between the pressing protrusion and the support protrusion.

19. The electrocautery surgical instrument of claim 18, wherein the actuation lever includes a plurality of pressing protrusions, wherein the plurality of pressing protrusions are disposed spaced apart at preset intervals.

20. The electrocautery surgical instrument of claim 12, wherein the actuation pulley is configured to receive the at least a portion of the rotational force of the actuation lever from the elastic member and transmit the at least a portion of the rotational force to the actuation wire when the actuation lever rotates.
